(19) United States
(12) Patent Application Publication
(10) Pub. No.: US 2006/0228412 A1
(43) Pub. Date: Oct. 12, 2006

(54) (-)-Hydroxycitric acid for delaying gastric emptying

(76) Inventors: Dallas L. Clouatre, Santa Monica, CA (US); James M. Dunn, Littleton, CO (US); Caroline Dunn, Littleton, CO (US)

Correspondence Address: FOLEY & LARDNER LLP, 111 HUNTINGTON AVENUE, 26TH FLOOR, BOSTON, MA, 02199-7610, US

(21) Appl. No.: 11/384,196

(22) Filed: Mar. 17, 2006

Related U.S. Application Data
(63) Continuation of application No. PCT/US04/30534, filed on Sep. 20, 2004.
Application No. PCT/US04/30534 is a continuation-in-part of application No. 10/666,667, filed on Sep. 20, 2003, now abandoned.

(51) Int. Cl. A61K 31/366; A61K 31/19; A61K 9/46

(52) U.S. Cl. 424/466; 514/574; 514/460

(57) ABSTRACT

The inventors have discovered that food and pharmaceutical compositions containing (−)-hydroxycitric acid, its salts, amides and esters can be employed for delaying gastric emptying and increasing receptive relaxation for preventing and treating diverse conditions. The invention provides for HCA-containing compound useful to delay gastric emptying and increase receptive relaxation for preventing and treating diverse conditions, e.g., stomach ulcers, portal hypertension, liver dysfunction, diabetes and obesity. The invention provides methods for delaying gastric emptying and increasing receptive relaxation in individuals. The invention also provides methods of preventing or treating disorders or conditions related to aberrant gastric emptying and receptive relaxation.

### FIELD OF THE INVENTION

The invention relates to the use of food and pharmaceutical compositions containing (−)-hydroxycitric acid (hereinafter, “HCA”), its salts, amides and esters for influencing glucagon-like peptides (GLP-1/2) and cholecystokinin (CCK), delaying gastric emptying and increasing gastric receptive relaxation for preventing and treating diverse conditions.

### BACKGROUND OF THE INVENTION

Receptive relaxation of gastrointestinal tissue, e.g., relaxation and expansion of the stomach to accommodate the volume of ingested food, prevents a change in total intragastric pressure that would otherwise be observed with an increase in gastrointestinal contents. Altered rates of gastric emptying often are accompanied by various health problems with the wall of the stomach itself or issues involving neighboring organs. Altered gastric emptying and accommodation are found with forms of portal hypertension, liver dysfunction and gastrointestinal ulcers, e.g., duodenal ulcer. Numerous medications, such as antibiotics (erythromycin, indomethacin, etc). and including even some diet drugs (e.g., Orlistat and other lipase inhibitors), that can accelerate gastric emptying. Surgery, such as for peptic ulcers, itself can lead to clinical dumping syndrome, as can other types of surgery performed on the stomach. Other factors or conditions that lead to acceleration of gastric emptying include obesity, high-energy density of food, fat intolerance, early stages of noninsulin-dependent diabetes mellitus, Zollinger-Ellison syndrome, and intermittent episodes in other forms of diabetes.

There remains a need for compounds to prevent or treat aberrant gastric emptying in a subject.

### SUMMARY OF THE INVENTION

The invention provides for hydroxycitrate-containing compounds (i.e., HCA-containing compounds) useful to delay gastric emptying and increase receptive relaxation for preventing and treating diverse conditions, e.g., stomach ulcers, portal hypertension, diabetes and obesity. In one embodiment, the hydroxycitrate-containing compound of the invention includes (−)-hydroxycitric acid, its salts, amides and esters can be employed for delaying gastric emptying and increasing receptive relaxation.

In one aspect, the invention provides a method for delaying gastric emptying and increasing receptive relaxation in individuals in need thereof which is comprised of administering orally an effective amount of (−)-hydroxycitric acid or one or more pharmaceutically effective and acceptable salts or derivatives of (−)-hydroxycitric acid selected from the group consisting of the free acid or its lactone, the alkali metal salts potassium or sodium HCA, the alkaline earth metal salts calcium or magnesium HCA, a mixture the alkali metal salts and/or the alkaline earth metal salts of HCA or some mixture of alkali metal salts and alkaline earth metal salts of HCA or in the form of therapeutically effective amide and/or ester derivatives of (−)-hydroxycitric acid. In one embodiment of the method of the invention, the (−)-hydroxycitric acid is supplied as a therapeutically effective amount as the free acid, its lactone or as one or more of the salts or other derivatives of the free acid and is delivered in a controlled release form. In one embodiment of the method of the invention, the salts or derivatives are administered orally as a tablet or capsule wherein the contents of said capsule or tablet further comprise sodium bicarbonate, calcium carbonate, or potassium bicarbonate for producing carbon dioxide gas on contact with the stomach liquids wherein the amount of sodium bicarbonate, calcium carbonate or potassium bicarbonate is sufficient to cause the breakup of the capsule or tablet thus releasing the salts or derivatives, but insufficient to cause distension of the stomach. In one embodiment of the method of the invention, the salts or derivatives are administered orally as a tablet or capsule wherein the contents of said capsule or tablet further comprise sodium bicarbonate or potassium bicarbonate plus alginic acid; also capsules or tables containing sodium or potassium alginate. In one embodiment of the method of the invention, the salts or derivatives are administered orally as dry packaged powders designed to be mixed with water or juice and consumed between meals or prior to meals. In one embodiment of the method of the invention, the salts or derivatives are administered orally and are further encased in materials selected from the group consisting of gelatin, tapioca, gums, pectins, inulin, cellulose derivatives, alginic acid, dextran and dextrin for inclusion in thick drinks, soft-center bars and candies, pudding snacks, jelly-like confections, “gummy” deliveries and liquid meal replacements. In one embodiment of the method of the invention, the salts or derivatives are administered orally in conjunction with materials selected from the group consisting of citric acid, sodium or potassium citrate, other citric acid salts, sodium propionate, propionic acid, gallic acid, propyl gallate; extracts of marigold (Calendula officinalis); escins and other compounds from Aesculus hippocastanum seeds; extracts of the fruit of Kochia scoparia, and the roots and other parts of Aralia elata; saponins, especially Theasaponin E1 from the seeds of the tea plant (Camellia sinensis L).; extracts from bay leaf (Laurus nobilis), especially costunolide and its active component, alpha-methylene-gamma-butyrolactone (alpha-MGBL); proteinase inhibitor extracts from potato and soybean sources; a variety of oleanolic acid glycosides from many sources; also herbal combinations such as one consisting of yerba mate, damiana and guarana.

In one embodiment, the invention provides a method for influencing glucagon-like peptides in individuals in need thereof which is comprised of administering orally an effective amount of (−)-hydroxycitric acid or one or more pharmaceutically effective and acceptable salts or derivatives of (−)-hydroxycitric acid selected from the group consisting of the free acid or its lactone, the alkali metal salts potassium or sodium HCA, the alkaline earth metal salts calcium or magnesium HCA, a mixture the alkali metal salts and/or the alkaline earth metal salts of HCA or some mixture of alkali metal salts and alkaline earth metal salts of HCA or in the form of therapeutically effective amide and/or ester derivatives of (−)-hydroxycitric acid.

In one embodiment of the method of the invention, the (−)-hydroxycitric acid is supplied as a therapeutically effective amount as the free acid, its lactone or as one or more of the salts or other derivatives of the free acid and is delivered in a controlled release form. In one embodiment of the method of the invention, the salts or derivatives are administered orally as a tablet or capsule wherein the contents of said capsule or tablet further comprise sodium bicarbonate, calcium carbonate, or potassium bicarbonate for producing carbon dioxide gas on contact with the stomach liquids wherein the amount of sodium bicarbonate, calcium carbonate or potassium bicarbonate is sufficient to cause the breakup of the capsule or tablet thus releasing the salts or derivatives, but insufficient to cause distension of the stomach. In one embodiment of the method of the invention, the method of claim 2 where the salts or derivatives are administered orally as a tablet or capsule wherein the contents of said capsule or tablet further comprise sodium bicarbonate or potassium bicarbonate plus alginic acid; also capsules or tables containing sodium or potassium alginate. In one embodiment of the method of the invention, the salts or derivatives are administered orally as dry packaged powders designed to be mixed with water or juice and consumed between meals or prior to meals. In one embodiment of the method of the invention, the salts or derivatives are administered orally and are further encased in materials selected from the group consisting of gelatin, tapioca, gums, pectins, inulin, cellulose derivatives, alginic acid, dextran and dextrin for inclusion in thick drinks, soft-center bars and candies, pudding snacks, jelly-like confections, “gummy” deliveries and liquid meal replacements. In one embodiment of the method of the invention, the salts or derivatives are administered orally in conjunction with materials selected from the group consisting of citric acid, sodium or potassium citrate, other citric acid salts, sodium propionate, propionic acid, gallic acid, propyl gallate; extracts of marigold (Calendula officinalis); escins and other compounds from Aesculus hippocastanum seeds; extracts of the fruit of Kochia scoparia, and the roots and other parts of Aralia elata; saponins, especially Theasaponin E1 from the seeds of the tea plant (Camellia sinensis L).; extracts from bay leaf (Laurus nobilis), especially costunolide and its active component, alpha-methylene-gamma-butyrolactone (alpha-MGBL); proteinase inhibitor extracts from potato and soybean sources; a variety of oleanolic acid glycosides from many sources; also herbal combinations such as one consisting of yerba mate, damiana and guarana.

In one embodiment, the invention provides a method for influencing cholecystokinin in individuals in need thereof which is comprised of administering orally an effective amount of (−)-hydroxycitric acid or one or more pharmaceutically effective and acceptable salts or derivatives of (−)-hydroxycitric acid selected from the group consisting of the free acid or its lactone, the alkali metal salts potassium or sodium HCA, the alkaline earth metal salts calcium or magnesium HCA, a mixture the alkali metal salts and/or the alkaline earth metal salts of HCA or some mixture of alkali metal salts and alkaline earth metal salts of HCA or in the form of therapeutically effective amide and/or ester derivatives of (−)-hydroxycitric acid. In one embodiment of the method of the invention, the (−)-hydroxycitric acid is supplied as a therapeutically effective amount as the free acid, its lactone or as one or more of the salts or other derivatives of the free acid and is delivered in a controlled release form. In one embodiment of the invention, the salts or derivatives are administered orally as a tablet or capsule wherein the contents of said capsule or tablet further comprise sodium bicarbonate, calcium carbonate, or potassium bicarbonate for producing carbon dioxide gas on contact with the stomach liquids wherein the amount of sodium bicarbonate, calcium carbonate or potassium bicarbonate is sufficient to cause the breakup of the capsule or tablet thus releasing the salts or derivatives, but insufficient to cause distension of the stomach. In one embodiment of the method of the invention, the salts or derivatives are administered orally as a tablet or capsule wherein the contents of said capsule or tablet further comprise sodium bicarbonate or potassium bicarbonate plus alginic acid; also capsules or tables containing sodium or potassium alginate. In one embodiment of the method of the invention, the salts or derivatives are administered orally as dry packaged powders designed to be mixed with water or juice and consumed between meals or prior to meals. In one embodiment of the method of the invention, the salts or derivatives are administered orally and are further encased in materials selected from the group consisting of gelatin, tapioca, gums, pectins, inulin, cellulose derivatives, alginic acid, dextran and dextrin for inclusion in thick drinks, soft-center bars and candies, pudding snacks, jelly-like confections, “gummy” deliveries and liquid meal replacements. In one embodiment of the method of the invention, the salts or derivatives are administered orally in conjunction with materials selected from the group consisting of citric acid, sodium or potassium citrate, other citric acid salts, sodium propionate, propionic acid, gallic acid, propyl gallate; extracts of marigold (Calendula officinalis); escins and other compounds from Aesculus hippocastanum seeds; extracts of the fruit of Kochia scoparia, and the roots and other parts of Aralia elata; saponins, especially Theasaponin E1 from the seeds of the tea plant (Camellia sinensis L).; extracts from bay leaf (Laurus nobilis), especially costunolide and its active component, alpha-methylene-gamma-butyrolactone (alpha-MGBL); proteinase inhibitor extracts from potato and soybean sources; a variety of oleanolic acid glycosides from many sources; also herbal combinations such as one consisting of yerba mate, damiana and guarana.

In one aspect, the invention provides a (−)-hydroxycitrate-containing composition, comprising (a) (−)-hydroxycitrate; (b) bicarbonate; and (c) starch; wherein the (−)-hydroxycitrate-containing composition decreases gastric emptying rate and increases receptive relaxation when orally administered to a subject. In one embodiment of the (−)-hydroxycitrate-containing composition the (−)-hydroxycitrate is selected from a group consisting of: (−)-hydroxycitrate free acid; (−)-hydroxycitrate salts; and (−)-hydroxycitrate derivatives, or any combination thereof. In one embodiment of the (−)-hydroxycitrate-containing composition, the (−)-hydroxycitrate is present from about 20 weight percent to about 80 weight percent of the (−)-hydroxycitrate-containing composition. In one embodiment of the (−)-hydroxycitrate-containing composition, the (−)-hydroxycitrate is present at a concentration from about 30 weight percent to about 70 weight percent of the (−)-hydroxycitrate-containing composition. In one embodiment of the (−)-hydroxycitrate-containing composition, the (−)-hydroxycitrate is present at a concentration at least about 50 weight percent of the (−)-hydroxycitrate-containing composition. In one embodiment of the (−) hydroxycitrate-containing composition, the bicarbonate is selected from a group consisting of: sodium bicarbonate; potassium bicarbonate; magnesium bicarbonate and calcium bicarbonate. In one embodiment of the (−)-hydroxycitrate-containing composition, the bicarbonate is present at a concentration from about 1 weight percent to about 20 weight percent of the (−)-hydroxycitrate-containing composition. In one embodiment of the (−)-hydroxycitrate-containing composition, the bicarbonate is present at a concentration from about 3 weight percent to about 10 weight percent of the (−)-hydroxycitrate-containing composition. In one embodiment of the (−)-hydroxycitrate-containing composition, wherein the bicarbonate is present at a concentration at least about 7 weight percent of the (−)-hydroxycitrate-containing composition. In one embodiment of the (−)-hydroxycitrate-containing composition, the starch is starch 1500. In one embodiment of the (−)-hydroxycitrate-containing composition, the starch is present at a concentration from about 2 weight percent to about 40 weight percent of the (−)-hydroxycitrate-containing composition. In one embodiment of the (−)-hydroxycitrate-containing composition, the starch is present at a concentration from about 2 weight percent to about 25 weight percent of the (−)-hydroxycitrate-containing composition. In one embodiment of the (−)-hydroxycitrate-containing composition, the starch is present at a concentration at least about 7.0 weight percent of the (−)-hydroxycitrate-containing composition. In one embodiment of the (−)-hydroxycitrate-containing composition, the composition further comprises malic acid. In one embodiment of the (−)-hydroxycitrate-containing composition, the malic acid is present at a concentration from about 10 weight percent to about 40 weight percent of the (−)-hydroxycitrate-containing composition. In one embodiment of the (−)-hydroxycitrate-containing composition, the malic acid is present at a concentration from about 15 weight percent to about 30 weight percent of the (−)-hydroxycitrate-containing composition. In one embodiment of the (−)-hydroxycitrate-containing composition, the malic acid is present at a concentration at least about 25 weight percent of the (−)-hydroxycitrate-containing composition. In one embodiment of the (−)-hydroxycitrate-containing composition, the composition further comprises alginic acid. In one embodiment of the (−)-hydroxycitrate-containing composition, the alginic acid is present at a concentration from about 5 weight percent to about 50 weight percent of the (−)-hydroxycitrate-containing composition. In one embodiment of the (−)-hydroxycitrate-containing composition, the alginic acid is present at a concentration from about 10 weight percent to about 40 weight percent of the (−)-hydroxycitrate-containing composition. In one embodiment of the (−)-hydroxycitrate-containing composition, the alginic acid is present at a concentration at least about 28 weight percent of the (−)-hydroxycitrate-containing composition. In one embodiment of the (−) hydroxycitrate-containing composition, the (−)-hydroxycitrate-containing composition is formulated as a soft gelatin encapsulation.

In one embodiment, the invention provides a (−)-hydroxycitrate-containing composition, comprising: (a) (−)-hydroxycitrate; (b) bicarbonate; (c) citric acid; (d) magnesium stearate; and (e) satialgine; wherein the (−)-hydroxycitrate-containing composition decreases gastric emptying rate and increases receptive relaxation when orally administered to a subject. In one embodiment of the (−)-hydroxycitrate-containing composition, the (−)-hydroxycitrate is selected from a group consisting of (−)-hydroxycitrate free acid; (−)-hydroxycitrate salts; and (−)-hydroxycitrate derivatives, or any combination thereof. In one embodiment of the (−)-hydroxycitrate-containing composition, the (−)-hydroxycitrate is present from about 20 weight percent to about 80 weight percent of the (−)-hydroxycitrate-containing composition. In one embodiment of the (−)-hydroxycitrate-containing composition, the (−)-hydroxycitrate is present at a concentration from about 30 weight percent to about 70 weight percent of the (−)-hydroxycitrate-containing composition. In one embodiment of the (−)-hydroxycitrate-containing composition, the (−)-hydroxycitrate is present at a concentration at least about 50 weight percent of the (−)-hydroxycitrate-containing composition. In one embodiment of the (−)-hydroxycitrate-containing composition, the bicarbonate is selected from a group consisting of: sodium bicarbonate; potassium bicarbonate; magnesium bicarbonate and calcium bicarbonate. In one embodiment of the (−)-hydroxycitrate-containing composition, the bicarbonate is present at a concentration from about 1 weight percent to about 20 weight percent of the (−)-hydroxycitrate-containing composition. In one embodiment of the (−)-hydroxycitrate-containing composition, the bicarbonate is present at a concentration from about 3 weight percent to about 10 weight percent of the (−)-hydroxycitrate-containing composition. In one embodiment of the (−)-hydroxycitrate-containing composition, the bicarbonate is present at a concentration at least about 14 weight percent of the (−)-hydroxycitrate-containing composition. In one embodiment of the (−)-hydroxycitrate-containing composition, the citric acid is present at a concentration from about 5 weight percent to about 40 weight percent of the (−)-hydroxycitrate-containing composition. In one embodiment of the (−)-hydroxycitrate-containing composition, the citric acid is present at a concentration from about 10 weight percent to about 30 weight percent of the (−)-hydroxycitrate-containing composition. In one embodiment of the (−)-hydroxycitrate-containing composition, the citric acid is present at a concentration at least about 14 weight percent of the (−)-hydroxycitrate-containing composition. In one embodiment of the (−)-hydroxycitrate-containing composition, the magnesium stearate is present at a concentration from about 0.01 weight percent to about 5 weight percent of the (−)-hydroxycitrate-containing composition. In one embodiment of the (−)-hydroxycitrate-containing composition, the magnesium stearate is present at a concentration from about 0.1 weight percent to about 2 weight percent of the (−)-hydroxycitrate-containing composition. In one embodiment of the (−)-hydroxycitrate-containing composition, the magnesium stearate is present at a concentration at least about 0.5 weight percent of the (−)-hydroxycitrate-containing composition. In one embodiment of the (−)-hydroxycitrate-containing composition, the satialgine is present at a concentration from about 5 weight percent to about 40 weight percent of the (−)-hydroxycitrate-containing composition. In one embodiment of the (−)-hydroxycitrate-containing composition, the satialgine is present at a concentration from about 10 weight percent to about 30 weight percent of the (−)-hydroxycitrate-containing composition. In one embodiment of the (−)-hydroxycitrate-containing composition, the satialgine is present at a concentration at least about 14 weight percent of the (−)-hydroxycitrate-containing composition. In one embodiment of the (−)-hydroxycitrate-containing composition, the (−)-hydroxycitrate-containing composition is formulated as a soft gelatin encapsulation.

In one embodiment, the invention provides a (−)-hydroxycitrate-containing composition, comprising: (a) (−)-hydroxycitrate; (b) mannitol; (c) aspartame; (d) magnesium stearate; and (e) satialgine; wherein the (−)-hydroxycitrate-containing composition decreases gastric emptying rate and increases receptive relaxation when orally administered to a subject. In one embodiment of the (−)-hydroxycitrate-containing composition, the (−)-hydroxycitrate is selected from a group consisting of: (−)-hydroxycitrate free acid; (−)-hydroxycitrate salts; and (−)-hydroxycitrate derivatives, or any combination thereof. In one embodiment of the (−)-hydroxycitrate-containing composition, the (−)-hydroxycitrate is present from about 20 weight percent to about 95 weight percent of the (−)-hydroxycitrate-containing composition. In one embodiment of the (−)-hydroxycitrate-containing composition, the (−)-hydroxycitrate is present at a concentration from about 30 weight percent to about 85 weight percent of the (−)-hydroxycitrate-containing composition. In one embodiment of the (−)-hydroxycitrate-containing composition, the (−)-hydroxycitrate is present at a concentration at least about 73 weight of the (−)-hydroxycitrate-containing composition. In one embodiment of the (−)-hydroxycitrate-containing composition, the mannitol is present at a concentration from about 1 weight percent to about 50 weight percent of the (−)-hydroxycitrate-containing composition. In one embodiment of the (−)-hydroxycitrate-containing composition, the mannitol is present at a concentration from about 10 weight percent to about 30 weight percent of the (−)-hydroxycitrate-containing composition. In one embodiment of the (−)-hydroxycitrate-containing composition, the mannitol is present at a concentration at least about 20 weight percent of the (−)-hydroxycitrate-containing composition. In one embodiment of the (−)-hydroxycitrate-containing composition, the satialgine is present at a concentration from about 0.01 weight percent to about 5 weight percent of the (−)-hydroxycitrate-containing composition. In one embodiment of the (−)-hydroxycitrate-containing composition, the satialgine is present at a concentration from about 0.1 weight percent to about 3 weight percent of the (−)-hydroxycitrate-containing composition. In one embodiment of the (−)-hydroxycitrate-containing composition, the satialgine is present at a concentration at least about 0.3 weight percent of the (−)-hydroxycitrate-containing composition. In one embodiment of the (−)-hydroxycitrate-containing composition, the aspartame is present at a concentration from about 0.01 weight percent to about 5 weight percent of the (−)-hydroxycitrate-containing composition. In one embodiment of the (−) hydroxycitrate-containing composition, the aspartame is present at a concentration from about 0.1 weight percent to about 3 weight percent of the (−)-hydroxycitrate-containing composition. In one embodiment of the (−)-hydroxycitrate-containing composition, the aspartame is present at a concentration at least about 0.6 weight percent of the (−)-hydroxycitrate-containing composition. In one embodiment of the (−)-hydroxycitrate-containing composition, the magnesium stearate is present at a concentration from about 0.01 weight percent to about 5 weight percent of the (−)-hydroxycitrate-containing composition. In one embodiment of the (−)-hydroxycitrate-containing composition, the magnesium stearate is present at a concentration from about 0.1 weight percent to about 2 weight percent of the (−)-hydroxycitrate-containing composition. In one embodiment of the (−)-hydroxycitrate-containing composition, the magnesium stearate is present at a concentration at least about 0.6 weight percent of the (−)-hydroxycitrate-containing composition. In one embodiment of the (−)-hydroxycitrate-containing composition, the composition further comprises food coloring. In one embodiment of the (−)-hydroxycitrate-containing composition, the food coloring is orange food coloring. In one embodiment of the (−)-hydroxycitrate-containing composition, the food coloring is present at a concentration from about 0.1 weight percent to about 10 weight percent of the (−)-hydroxycitrate-containing composition. In one embodiment of the (−)-hydroxycitrate-containing composition, the food coloring is present at a concentration from about 1 weight percent to about 5 weight percent of the (−)-hydroxycitrate-containing composition. In one embodiment of the (−)-hydroxycitrate-containing composition, the food coloring is present at a concentration at least about 2 weight percent of the (−)-hydroxycitrate-containing composition. In one embodiment of the (−)-hydroxycitrate-containing composition, the composition further comprises food flavoring. In one embodiment of the (−)-hydroxycitrate-containing composition, the food flavoring is orange food flavoring. In one embodiment of the (−)-hydroxycitrate-containing composition, the food flavoring is present at a concentration from about 0.1 weight percent to about 10 weight percent of the (−)-hydroxycitrate-containing composition. In one embodiment of the (−)-hydroxycitrate-containing composition, the food flavoring is present at a concentration from about 1 weight percent to about 5 weight percent of the (−)-hydroxycitrate-containing composition. In one embodiment of the (−)-hydroxycitrate-containing composition, the food flavoring is present at a concentration at least about 2.5 weight percent of the (−)-hydroxycitrate-containing composition. In one embodiment of the (−)-hydroxycitrate-containing composition, the (−)hydroxycitrate-containing composition is formulated as a soft gelatin encapsulation.

In one embodiment, the invention provides a method of decreasing the rate gastric emptying and increase receptive relaxation in a subject, the method comprising administering to a subject in which a decreased gastric emptying rate and an increase in receptive relaxation is desired an HCA-containing composition of the invention in an amount sufficient to decrease the rate of gastric emptying and increase receptive relaxation in the subject.

### DETAILED DESCRIPTION

I. Definitions

A “subject,” as used herein, is preferably a mammal, such as a human, but can also be an animal, e.g., domestic animals (e.g., dogs, cats and the like), farm animals (e.g., cows, sheep, pigs, horses and the like) and laboratory animals (e.g., rats, mice, guinea pigs and the like).

An “effective amount” of an HCA-containing compound of the invention, as used herein, is a quantity sufficient to achieve a desired therapeutic and/or prophylactic effect, for example, an amount which results in the prevention of or a decrease in the symptoms associated with a disease, disorder or condition that is being treated, e.g., obesity, ulcer, diabetes, portal hypertension. The amount of an HCA-containing composition of the invention administered to the subject will depend on the type and severity of the disease, disorder or condition, and on the characteristics of the individual, such as general health, age, sex, body weight and tolerance to drugs. It will also depend on the degree, severity and type of disease. The skilled artisan will be able to determine appropriate dosages depending on these and other factors. Typically, an effective amount of the HCA-containing compound of the invention, sufficient for achieving a therapeutic or prophylactic effect, range from about 0.000001 mg per kilogram body weight per day to about 10,000 mg per kilogram body weight per day. Preferably, the dosage ranges are from about 0.0001 mg per kilogram body weight per day to about 100 mg per kilogram body weight per day. A common dosage range is between 300-5,000 mg per day. Another common dosage range is between 1,000-4,000 mg per day. A common daily dose is 3,000 mg per day. The HCA-containing compound of the invention can also be administered in combination alone, or with one or more additional therapeutic compounds.

The HCA-containing compound of the invention can also be administered in combination alone, or with one or more additional therapeutic compounds. The compounds of the present invention are useful as dietary supplements.

The references cited in this application are incorporated by reference herein in their entireties.

II. General

(−)-Hydroxycitric acid (HCA) is a naturally-occurring acid found in the fruit of members of the plant genus Garcinia. Free HCA, calcium, magnesium and potassium salts of HCA (i.e., hydroxycitrates, also referred to as HCA) and poorly characterized mixtures of two or more of these minerals have been sold in the American market. Calcium HCA as well as double-metal HCA compositions containing both calcium HCA and sodium HCA (i.e., calcium/sodium salts) were sold as early as 1993. Most of the commercial preparations of HCA sold to date consist of calcium salts of varying degrees of purity or, more recently, poorly characterized mixtures of calcium HCA and potassium HCA salts. The physiological effects of HCA have been investigated for more than forty years.

HCA can affect the metabolic functions of mammals, including humans. HCA, as well as several synthetic derivatives of citric acid, can inhibit the production of fatty acids from carbohydrates, suppress appetite, and inhibit weight gain (Sullivan et al., Am. J. Clin. Nutr. 1977; 30: 767). Numerous other benefits have been attributed to the use of HCA, including, but not limited to, an increase in the metabolism of fat stores for energy and an increase in thermogenesis (the metabolism of energy sources to produce body heat in an otherwise wasteful cycle). HCA and its derivatives were not known to affect gastric emptying rate or receptive relaxation.

The present invention identifies HCA, its salts, amides and esters as modulators of gastric emptying rate and/or receptive relaxation in mammals, e.g., delay gastric emptying or increasing receptive relaxation. In one aspect, the present invention provides a new methods for the use of HCA-containing compounds to modulate gastric emptying rate and/or receptive relaxation in mammals. The invention identifies HCA, its salts, amides and esters as useful for delaying gastric emptying and for increasing receptive relaxation and thus can be utilized for preventing and/or treating conditions or disorders related to aberrant gastric emptying. In one embodiment, at least one HCA-containing compound is combined with other food and administered to a subject to delay gastric emptying and/or to increase receptive relaxation. In one embodiment, at least one HCA-containing compound is formulated as a pharmaceutical compositions and administered to a subject to delay gastric emptying and/or to increase receptive relaxation.

Further objects and advantages include the employment of HCA in conditions such as presinusoidal portal hypertension, liver cirrhosis, duodenal ulcer, dumping syndrome, accelerated gastric emptying due to drugs (antibiotics, lipase inhibitors, etc)., rapid gastric emptying due to pre-diabetic and diabetic conditions, and various other circumstances described above. These objects and advantages are not derived from the anorectic actions commonly claimed for the use of HCA as an anti-obesity agent, but rather depend upon other physiological mechanisms.

Moreover, these objects and advantages do not require adherence to current dosage regimens. Current recommendations for the use of HCA require that it be ingested either two or three times per day 30 to 60 minutes prior to meals for weight loss. However, such a regimen may be of little benefit in conditions such as those involving duodenal ulcers or gastric lesions where extended residence time for HCA in contact with the stomach is desirable. Similarly, current recommendations for the use of HCA may not benefit those suffering from drug- or surgery-induced dumping or rapid gastric emptying.

The present invention improves and expands the use of HCA in the field of bariatrics. HCA can now be used to overcome at least some of the side effects of weight loss drugs such as Orlistat. Through the use of the present invention it is also possible to overcome the primary impediment to the successful employment of HCA for weight loss during the first two months of use and achieve consistent results in humans, something not evidenced in published clinical trials performed in the United States and Europe.

Altered gastric emptying and accommodation characterize a number of disease conditions. Gastric accommodation to distension from an influx of food, also called receptive relaxation, can prevent the change in total intragastric pressure despite an increase in stomach contents. Altered rates of gastric emptying often are accompanied by various health problems with the wall of the stomach itself or issues involving neighboring organs. Thus, there appear to be links from altered gastric emptying rates to conditions as seemingly diverse as stomach ulcers and portal hypertension, as well as more to be expected conditions, such diabetes and obesity.

That diverse conditions are linked to altered rates of gastric emptying reflects the fact that gastric motility is controlled, at least in part, by vagal inhibitory neurons, various postganglionic nerves and a variety of endocrine and non-endocrine compounds. Among the proposed compounds are acetylcholine, norepinephrine, secretin, glucagon, motilin, glucagon-like peptides,

peptide YY and serotonin. Unfortunately, the evidence for most of these remains unsettled as there inconsistencies among study results in the field. Hence, proposing mechanisms often is remote from demonstrating practical methods for delaying gastric emptying. For instance, although serotonin is produced and released by a number of gastrointestinal neurons, the use of compounds that powerfully influence serotonin reuptake or otherwise act as agonists in clinical experiments has failed to significantly affect gastric emptying.

Accelerated gastric emptying and a reduction of gastric accommodation are symptoms often found in hypertension caused by an obstacle to portal blood circulation. Blockages of this sort provoke congestion of the stomach wall and the intestine as well as functional disorders in these viscera. (Aprile L R, Meneghelli U G, Martinelli A L, Monteiro C R. Gastric motility in patients with presinusoidal portal hypertension. Am J Gastroenterol. 2002 December;97(12):3038-44). Gastric emptying in liver cirrhosis may similarly be accelerated. This symptom in cirrhosis is primarily found with smaller and more liquid meals, which is unfortunate because the emptying of larger meals in these patients, which tends to be either more or less normal or even delayed, remains improperly coordinated with bile release, which is, again, inadequate. (Acalovschi M, Dumitrascu D L, Csakany I. Gastric and gall bladder emptying of a mixed meal are not coordinated in liver cirrhosis—a simultaneous sonographic study. Gut. 1997 March;40(3):412-7).

Ulcers constitute another set of conditions that are characterized by dysregulations in gastric emptying. Gastric emptying is rapid in patients with proximal gastric ulcer due to accelerated proximal evacuation. Similarly, rapid emptying is seen in duodenal ulcer patients and is considered to be due to accelerated emptying in both the proximal stomach and the antrum. However, emptying is delayed in patients with distal gastric ulcer due to reduced emptying in the antrum. Gastric emptying in the healing stage is closer to that found in healthy subjects than in patients with active-stage ulcer.

Of common ulcers, duodenal ulcers most likely would benefit from delaying gastric emptying and a reduction in the excessive stomach acid entering the duodenum. Recent research bears this out and indicates that drug-induced ulcers and non-H. pylori ulcers may be more common than once thought. “It is increasingly recognized that different causes of ulcers coexist in a given patient, confounding determination of the exact cause of the ulcer. For example, in infected patients with ulcers who also are using nonsteroidal anti-inflammatory drugs (NSAIDs), it is not possible to establish the ulcer's cause. Moreover, recent studies in the United States in infected patients with duodenal ulcers who were treated with various regimens to prove their efficacy in eradicating Helicobacter pylori (H. pylori) and preventing ulcer recurrence found that approximately 20% of patients suffered an ulcer recurrence despite successful H. pylori eradication. The infection dearly did not cause their ulcers but was originally thought to have done so. Thus, as many as one-fifth of patients with ulcers may have the cause falsely attributed to H. pylori infection. When this number is added to that of ulcer patients who are H. pylori-negative upon original presentation—at least 20% in other recent U.S. studies—it is evident that the proportion of non-H. pylori ulcer patients is larger than originally believed. This proportion is likely to increase with the declining incidence of H. pylori infection. Other causes of ulcers include the use of aspirin and NSAIDs (which may be surreptitious), hypersecretory states, Crohn's disease, and patients with “idiopathic” ulcers. Patients with “idiopathic” ulcers are characterized by postprandial hypersecretion of acid and hypergastrinemia with accelerated gastric emptying.” (Freston J W. Helicobacter pylori-negative peptic ulcers: frequency and implications for management. J Gastroenterol. 2000;35 Suppl 12:29-32).

Among the possible contributory causes of ulcers are recent diet drugs. Orlistat in particular has been shown to speed gastric emptying while at the same time increasing postprandial gastric acidity. This is the pattern already noted in duodenal ulcers. Inasmuch as lipase release plays an important role in reducing gastric acidity and in inhibiting gastric emptying (Borovicka J, et al. Role of lipase in the regulation of postprandial gastric acid secretion and emptying of fat in humans: a study with orlistat, a highly specific lipase inhibitor. Gut. 2000 June;46(6):774-81), it is likely that other lipase inhibitors, as well, may contribute to seldom recognized side effects, such as challenges to the integrity of the duodenum.

In contrast with Orlistat, at least one item used for weight loss actually protects against ulcer formation. Garcinia cambogia extract has been tested for its anti-ulcerogenic effect. Oral pretreatment or rats with Garcinia cambogia fruit extract (1 g/kg body wt/day) for 5, 10 or 15 days protected the gastric mucosa against the damage induced by indomethacin (20 mg/kg body wt). The volume and acidity of the gastric juice decreased in the pretreated animals. The glycoprotein levels of the gastric contents were decreased in the untreated rats, but remained at near normal levels in the pretreated animals. Likewise, protein was elevated in the gastric juice of untreated rats but, again, remained near normal levels in the pretreated rats. The extract was able to decrease the acidity and to increase the mucosal defense in the gastric areas. (Mahendran P, Vanisree A J, Shyamala Devi C S. The antiulcer activity of Garcinia cambogia extract against indomethacin-induced gastric ulcer in rats, Phytother Res. 2002 February;16(1):80-3). Similar protective effects have been reported against alcohol-induced ulceration (Mahendran P, Sabitha K E, Devi C S. Prevention of HCl-ethanol induced gastric mucosal injury in rats by Garcinia cambogia extract and its possible mechanism of action. Indian J Exp Biol. 2002 January;40(1):58-62).

As can be seen from the foregoing, accelerated gastric emptying is associated with a variety of medical conditions. Altered gastric emptying and accommodation are found with forms of hypertension, liver dysfunction and gastrointestinal ulcers. Numerous medications, such as antibiotics (erythromycin, indomethacin, etc). and including even some diet drugs, can accelerate gastric emptying. Surgery, such as for peptic ulcers, itself can lead to clinical dumping syndrome, as can other types of surgery performed on the stomach. “The factors or conditions that lead to normal acceleration of gastric emptying include coffee, cigarette smoking, obesity, high-energy density of food, fat intolerance, and hypertension. The conditions that can lead to abnormal acceleration of gastric emptying and symptoms mimicking EDS include idiopathic etiology, subtotal gastrectomy, early stages of noninsulin-dependent diabetes mellitus, Zollinger-Ellison syndrome, and duodenal ulcer.” (Singh A, Gull H. Singh R J. Clinical significance of rapid (accelerated) gastric emptying. Clin Nucl Med. 2003 August;28(8):658-62).

HCA Studies

Sullivan and co-workers consistently maintained that HCA does not influence gastric emptying (Sullivan C, Triscari J. Possible interrelationhip between metabolite flux and appetite. In D. Novin, W. Wyriwicka and G. Bray, eds., Hunger Basic Mechanisms and Clinical Implications (New York: Raven Press, 1976) 115-125; Sullivan C, Triscari J. Metabolic regulation as a control for lipid disorders. I. Influence of HCA on experimentally induced obesity in the rodent. Am J Clin Nutr. 1977 May;30(5):767-76; Sullivan C, Triscari J. Novel pharmacological approaches to the treatment of obesity. In George A. Bray, ed., Recent Advances in Obesity Research: II (Westport, Conn.: Technomic Publishing Co., 1977) 442-452; Sullivan A C, Dairman W, Triscari J. (−−)-threo-Chlorocitric acid: a novel anorectic agent. Pharmacol Biochem Behav. 1981 August;15(2):303-10; Sullivan, A. C., J. Triscari and L. Cheng. Appetite regulation by drugs and endogenous substances. In Myron Winick, ed., Nutrition and Drugs (New York: Wiley & Sons, 1983), 139-167. Also published as Sullivan A C, Triscari J, Cheng L. Appetite regulation by drugs and endogenous substances. Curr Concepts Nutr. 1983;12:139-67; Sullivan, Ann C. and J. Triscari. Pharmacologic approaches to the regulation of metabolism and obesity. In Jules Hirsch and Theodore B. Van Itallie, eds., Recent Advances in Obesity Research: IV (London: John Libbey, 1983) 196-207; Sullivan A C, Gruen R K. Mechanisms of appetite modulation by drugs. Fed Proc. 1985 January;44(1 Pt 1):139-44; Triscari J, Sullivan A C. Studies on the mechanism of action of a novel anorectic agent, (−−)-threo-chlorocitric acid. Pharmacol Biochem Behav. 1981 August;15(2):311-8). It should be noted that researchers Sullivan and Triscari were aware at least as early as 1976 that duodenal glucose receptors regulate appetite, yet they never made the connection with HCA. This position was borne of the conviction that all of the appetite-suppressing effects of the compound arise from its impact upon the liver and the activation of sugar-sensing neurons. Tests to establish the appetite suppressing effects of HCA showed that a single large oral dose or two divided oral doses totaling approximately one-fourth the size of the single dose resulted in a 10% or greater reduction in food consumption in experimental animals fed a high-sugar diet. (Minimum doses were 2.63 mmoles/kg once per day or 0.33 mmoles/kg twice per day either one hour before meals or four hours after, but not after the last meal of the day). This result continued over many weeks with the chronic ingestion of HCA. The appetite control mechanism of HCA was not thought to involve any conditioned aversion to food, i.e., HCA does not alter taste, cause gastric distress or illness, etc. Rather, this control was thought to stem from the increased production of glycogen and/or stimulation of glucoreceptors in the liver, either of which results in early satiety through signals sent to the brain via the vagus nerve.

It has now been demonstrated experimentally that the position that HCA suppresses appetite through vagal afferents associated with the liver is not correct. In an animal trial in which the hepatic branch of the vagus was severed (hepatic branch vagotomy), there was no significant effect found with this surgery in comparison with controls. (Leonhardt M, Langhans W. Effect of hydroxycitrate on food intake and body weight regain in rats after hepatic branch vagotomy or sham vagotomy. Society for the Study of Ingestive Behavior, Annual Meeting 2001).

Very recent papers have cast no more light on the anorectic effects of HCA. One research team that looked into the effects of HCA on serum leptin and insulin levels in mice had no new insights other than to suggest that HCA displays leptin-like activity, a point that the inventors made several years ago and for which we hold U.S. Pat. No. 6,476,071. (Hayamizu K, et al. Effect of Garcinia cambogia extract on serum leptin and insulin in mice. Fitoterapia. 2003 April;74(3):267-73). Another paper that directly confronts the issue says, “the anorectic mechanism of HCA is unknown.” (Leonhardt M, Langhans W. Hydroxycitrate has long-term effects on feeding behavior, body weight regain and metabolism after body weight loss in male rats. J Nutr. 2002 July;132(7):1977-82).

Yet another recent study suggests that HCA acts by means of influencing serotogenic mechanisms. This conclusion appears to be based on in vitro data, to wit “[HCA] can inhibit [3H]-5-HT uptake (and also increase 5-HT availability) in isolated rat brain cortical slices in a manner similar to that of SRRIs, and thus may prove beneficial in controlling appetite, as well as treatment of depression, insomnia, migraine headaches and other serotonin-deficient conditions.” (Ohia S E, et al. Safety and mechanism of appetite suppression by a novel hydroxycitric acid extract (HCA-SX). Mol Cell Biochem. 2002 September;238(1-2):89-103). These conclusions and speculations do not touch on gastric emptying.

Some early preliminary work showed that labeled ¹⁴C attached to HCA found its way into the brain. (Sullivan C, Triscari J. Metabolic regulation as a control for lipid disorders. I. Influence of HCA on experimentally induced obesity in the rodent. Am J Clin Nutr. 1977 May;30(5):767-76). However, work published by the same authors at a later date indicated otherwise. “Hydroxycitrate, chlorocitrate, and epoxyaconitate, compounds that are structurally similar to the tricarboxylic acid cycle intermediate citric acid, but that differ markedly in biochemical activity, have recently been evaluated in animals for effects on appetite. Because neither these compounds nor their metabolites enter the brain, their primary effects on food intake occur by peripheral mechanisms.” (Sullivan A C, Gruen R K Mechanisms of appetite modulation by drugs. Fed Proc. 1985 January;44(1 Pt 1):139-44). Again, it is well known that peripheral serotonin is metabolized virtually entirely peripherally. Indeed, this fact led to great concern when the compound 5-HTP (5-hydroxytryptophan extracted from the seeds of Griffonia simplicifolia) was first introduced as a dietary supplement Moreover, even in the rat brain slices, it is likely that citrate would have yielded the same results as did HCA inasmuch as this was found to be the case in earlier brain slice experiments looking at acetylcholine production. (Tucek S, Dolezal V, Sullivan A C. Inhibition of the synthesis of acetylcholine in rat brain slices by (−)-hydroxycitrate and citrate. J. Neurochem. 1981 April;36(4):1331-7). In any event, the same mistakes are made by the same authors in Ohia, Sunny E. et al., Jun. 26, 2003, United States Patent Application 20030119913 (also available as WO 03/053454). Moreover, even had Ohia, et al. not relied on rat brain slices, but rather on direct blood tests in humans, their suggestion as the anorectic impact of serotonin from the ingestion of HCA would not have had significance with regard to gastric emptying. Several sets of researchers have demonstrated that serotonin, either locally or centrally, is not likely the major agent in the control of gastric emptying (Chial H J, et al. Selective effects of serotonergic psychoactive agents on gastrointestinal functions in health. Am J Physiol Gastrointest Liver Physiol. 2003 January;284(1):G130-7). (Hansen L, Holst J J. The effects of duodenal peptides on glucagon-like peptide-1 secretion from the ileum. A duodeno—ileal loop? Regul Pept. 2002 Dec. 31;110(1):39-45). While there is evidence of the expression of 5-HT receptors by extrinsic duodenal afferents, both vagal and spinal, that can be blocked by some (but not all) antagonists to reduce the inhibition of gastric emptying induced by glucose and mannitol, attempts to increase this gastric inhibitory effect via 5-HT agonists have not met with success. Indeed, increased levels of 5-HT in the gut tend to be associated not with delayed gastric emptying, but rather with irritable bowel syndrome (IBS) and diarrhea. The recommendation in such cases are 5-HT antagonists. (Chey W D. Tegaserod and other serotonergic agents: what is the evidence? Rev Gastroenterol Disord. 2003;3 Suppl 2:S35-40).

Very recent reviews of the chemistry and biochemistry have added little insight to the anorectic and weight loss actions of HCA. One such review, following recent research, argues that the inhibition of ATP:citrate lyase by HCA markedly diminishes the cellular pool of malonyl-CoA, indicating that citrate was the major substrate for the malonyl-CoA precursor, that is, cytosolic acetyl-CoA. There is sufficient evidence that because HCA inhibits ATP:citrate lyase, it also acts to limit the pool of cytosolic acetyl-CoA, the precursor of malonyl-CoA. This type of regulation of the malonyl-CoA level may affect the signaling of fuel status in hypothalamic neurons regulating feeding behavior. In the opinion of this review, these findings lend support to the theory that HCA may represent a biochemical target for the control of appetite/feeding behavior and body weight, by acting at the metabolic level and not directly via the central nervous system as do classical appetite depressants. (Jena B S, Jayaprakasha G K, Singh R P, Sakariah K K. Chemistry and biochemistry of (−)-hydroxycitric acid from Garcinia. J Agric Food Chem. 2002 Jan. 2;50(1):10-22). This review does not consider issues of gastric emptying or short-term actions by HCA on gastric motility. Presently, “[T]the mechanism of the feeding suppressive effect of HCA has still to be identified.” (Leonhardt M, Hrupka B J, Langhans W. Subdiaphragmatic vagal deafferentation fails to block the anorectic effect of hydroxycitrate. Physiol Behav. 2004 Sep. 15;82(2-3):263-8.)

That there are quite serious difficulties with the present use of HCA as a weight loss agent is obvious from readily available published data. US and European trials have cast doubt on its efficacy. In part, this may be due to the salts used in the trials. Of the readily available forms of HCA, only the potassium and sodium salts of HCA are absorbed well enough to be effective agents at tolerable levels of ingestion. Calcium salts of HCA are markedly inferior to the potassium salt, and even including calcium as part of a potassium salt to form a double metal salt which is more workable than is the hygroscopic pure potassium salt at the same time significantly reduces efficacy. Several derivatives of HCA may also be active and effective. (U.S. Pat. Nos. 3,993,668; 3,919,254; 3,767,678). However, liquid forms of HCA currently in use are irritating to the digestive system, depending upon the dose, and may cause an elevation of stress hormones as a result. Researchers have found that animals given high doses of the liquid form of the compound orally exhibit stress behavior. (Ishihara K, Oyaizu S, Onuki K, Lim K, Fushiki T. Chronic HCA administration spares carbohydrate utilization and promotes lipid oxidation during exercise in mice. J Nutr. 2000 December;130(12):2990-5). Similarly, the ethylenediamine salts of HCA used in some of the later research performed by Sullivan and coworkers are known to be irritating and even toxic, properties which are due to the ethylenediamine ligand and not to the HCA.

All of the more recent and more thorough clinical trials on HCA not only have failed to produce appetite suppression, but also have produced trends toward weight gain in some instances. (Heymsfield S B, Allison D B, Vasselli J R, Pietrobelli A, Greenfield D, Nunez C. Garcinia cambogia (hydroxycitric acid) as a potential antiobesity agent a randomized controlled trial. JAMA. 1998;280:1596-1600; Mattes R D, Bormann L. Effects of (−)-hydroxycitric acid on appetitive variables. Physiol Behav. 2000 Oct. 1;71(1-2):87-94). Although they did not pursue the matter thoroughly, two Roche researchers in 1977 showed that HCA in the cytosol of the cell will activate acetyl CoA carboxylase similarly to the citrate it resembles. The effect of this property is that in diets which supply a source of acetyl CoA to the cytosol other than via citrate derived from the mitochondria, which means diets containing appreciable amounts of fat or alcohol as opposed to diets consisting almost exclusively of carbohydrates, HCA may increase the synthesis of fats and weight gain. (Triscari J, Sullivan A C. Comparative effects of HCA and (+)-allo-hydroxycitrate on acetyl CoA carboxylase and fatty acid and cholesterol synthesis in vivo. Lipids April 1977;12(4): 357-363). Patents which have been granted to date for the employment of HCA as an antiobesity agent (U.S. Pat. Nos. 3,764,692; 5,626,849; 5,783,603; 5,914,326 and others proposing the use of HCA as an adjunctive ingredient) have not indicated any awareness of its paradoxical effects, effects that have led to either null or negative results in the major clinical trials with HCA up to the point of this writing.

HCA actually exerts several quite distinct effects and ‘reverse effects’ can be triggered by dose amounts and/or dosing patterns that are inappropriate to match diet and other factors. The present invention discloses that HCA delays gastric emptying. Clouatre and coworkers findings that the weight loss attributable to lessened food intake can be distinguished analytically from weight loss which appears related to changes in metabolism and that the anorectic effects of HCA do not normally last beyond approximately 7 weeks have been described elsewhere. Clouatre and coworkers have further noted that higher fat (and alcohol) diets require higher dosages of HCA. Moreover, inadequate dosages of HCA can lead to weight gain. (See U.S. Pat. No. 6,476,071 and also U.S. patent application Ser. No. 10/616,321 entitled “Treating Cachexia and Excessive Catabolism with (−)-Hydroxycitric Acid.”)

Sullivan and coworkers maintained that the minimum effective doses of HCA in rats on a low fat diet (using trisodiumhydroxycitrate as the salt) are 2.63 mmoles/kg once per day or 0.33 mmoles/kg twice per day (Sullivan C, Triscari J. Possible interrelationhip between metabolite flux and appetite. In D. Novin, W. Wyriwicka and G. Bray, eds., Hunger: Basic Mechanisms and Clinical Implications (New York: Raven Press, 1976) 115-125; Sullivan C, Triscari J. Metabolic regulation as a control for lipid disorders. I. Influence of HCA on experimentally induced obesity in the rodent. Am J Clin Nutr. 1977 May;30(5):767-76; Sullivan C, Triscari J. Novel pharmacological approaches to the treatment of obesity. In George A. Bray, ed., Recent Advances in Obesity Research: II (Westport, Conn.: Technomic Publishing Co., 1977) 442-452; Sullivan A C, Dairman W, Triscari J. (−−)-threo-Chlorocitric acid: a novel anorectic agent. Pharmacol Biochem Behav. 1981 August;15(2):303-10; Sullivan, A. C., J. Triscari and L. Cheng. Appetite regulation by drugs and endogenous substances. In Myron Winick, ed., Nutrition and Drugs (New York: Wiley & Sons, 1983), 139-167. Also published as Sullivan A C, Triscari J, Cheng L. Appetite regulation by drugs and endogenous substances. Curr Concepts Nutr. 1983;12:139-67; Sullivan, Ann C. and J. Triscari. Pharmacologic approaches to the regulation of metabolism and obesity. In Jules Hirsch and Theodore B. Van Itallie, eds., Recent Advances in Obesity Research: IV (London: John Libbey, 1983) 196-207; Sullivan A C, Gruen R K. Mechanisms of appetite modulation by drugs. Fed Proc. 1985 January;44(1 Pt 1):139-44; Triscari J, Sullivan A C. Studies on the mechanism of action of a novel anorectic agent, (−−)-threo-chlorocitric acid. Pharmacol Biochem Behav. 1981 August;15(2):311-8). When added to food, the typical dosage used by Sullivan and coworkers was 52.6 mmol/kg feed. All subsequent individuals and groups working with the compound accepted that it must be given either as one extremely massive dose or, preferably, as two or three smaller doses delivered 30 to 60 minutes prior to meals. The appetite control mechanism of HCA was said to stem from the increased production of glycogen and/or stimulation of glucoreceptors in the liver, either of which results in satiety through signals sent to the brain via the hepatic branch of the vagus nerve. As noted above, Sullivan and coworkers, over a period of many years repeatedly maintained that HCA does not influence gastric emptying.

The inventors, however, realized quite early that the procedures of Sullivan and coworkers and explanations do not fit the observable data regarding HCA. Sullivan and coworkers claimed that glucoreceptors in the liver become more active because of HCA and that there must be a further step of signaling the brain. However, this suggests that there should be a considerable time lag before appetite suppression appears inasmuch as food must exit the stomach and glucose must reach the liver before an effect appears. To the contrary, the inventors observed that under certain conditions the anorectic effect of HCA appears extremely rapidly. This is in line with the actions of cholecystokinin, glucagon-like peptide (GLP-1) and/or other regulators of gastric emptying, but it is not typical of serotogenic regulation.

Sullivan and coworker's position on HCA implied that the glucoreceptors must be “primed” by a previous meal in order for HCA to work well—no glycogen, no anorexia. To the contrary, the inventors found that such priming is not necessary. Although no priming is necessary, a “preload” is. This means that there must be food or volume in the stomach for HCA to work, as one would expect with an inhibitor of gastric emptying.

Whereas Sullivan and coworkers focused on the putative role of the liver in the satiety associated with HCA, the inventors were more impressed by the fact that de novo lipogenesis also occurred in tissues of the small intestine. This suggests that just as there are early sensing glucoreceptors in the duodenal mucosa which activate glucagon like peptide-1 (GLP-1) upon saturation with glucose and certain other sugars, one might expect that the presence of HCA to lead to the release of GLP-1 inasmuch as HCA to the cellular machinery looks like the citrate that is generated from excess glucose.

Sullivan and coworkers performed experiments in which the ventromedial hypothalamus (VMH, the so-called satiety center) was destroyed, yet HCA nevertheless maintained its appetite suppression. (Sullivan C, Triscari J. Metabolic regulation as a control for lipid disorders. I. Influence of HCA on experimentally induced obesity in the rodent. Am J Clin Nutr. 1977 May;30(5):767-76). The widely accepted theory is that the obese animal eats more because it releases less of the satiety-inducing neurotransmitter serotonin in the hypothalamus. This experiment indicated that HCA a) does not require an intact VMH and b) probably does not require the actions of serotonin in the brain.

Initially, studies by Sullivan and coworkers tied weight loss and decreased food consumption together, and it later only partially retreated from this stance. However, the data showed that at the end of 80 days, there was a 4% net reduction in food intake compared with controls, yet a 78% reduction in weight gain. (Sullivan C, Triscari J. Metabolic regulation as a control for lipid disorders. I. Influence of HCA on experimentally induced obesity in the rodent. Am J Clin Nutr. 1977 May;30(5):767-76). Moreover, in a pair-feeding study, the HCA-fed rats gained substantially less weight than did controls limited to the same food intake. (Greenwood M R, Cleary M P, Gruen R, Blase D, Stem J S, Triscari J. Sullivan A C. Effect of (−)-hydroxycitrate on development of obesity in the Zucker obese rat. Am J. Physiol. 1981 January;240(1):E72-8). The inventor's animal trials demonstrated that the reduction in food intake was not tightly linked to a reduction in weight gain.

Human trials have yielded results that indicate clearly that the appetite suppression found with HCA is only weakly related to weight loss. On the one hand, in a trial published in 2002, although food intake decreased 15-30%, there was no significant weight loss over 2 weeks. (Westerterp-Plantenga M S, Kovacs E M. The effect of (−)-hydroxycitrate on energy intake and satiety in overweight humans. Int J Obes Relat Metab Disord. 2002 June;26(6):870-2). In this case, the ingestion of 300 mg three times daily HCA from SuperCitriMax potassium (16%) calcium (11%) hydroxycitrate led to only a trend toward weight loss despite the very large decrease in caloric intake. U.S. Pat. No. 6,476,071, however, disclosed that ingesting too little HCA can even cause weight gain, probably due to the activation of acetyl-CoA carboxylase. The delivery via tomato juice in this study is very important. This juice is acid, hence even the calcium salt of HCA dissolves fully in it, yet the juice does not contain components that rapidly bind to the HCl. Moreover, tomato juice supplies adequate sugars to activate gut responses and the juice is extremely rich in potassium—much more so than, say, orange juice. As has also been disclosed by Clouatre and coworkers in other patents (e.g., U.S. Pat. Nos. 6,447,807 and 6,476,071), the potassium/calcium salt of HCA is not well absorbed in the small intestine and therefore the metabolic effect of SuperCitrimax, as is true of all similar calcium and potassium-calcium salts of HCA, is weak in comparison with effect of a fully reacted potassium salt. The 2-week period of the Westerterp-Plantenga study cited above was inadequate for the metabolic intervention to manifest.

On the other hand, 1,200 mg HCA daily given as tablets (2×400 mg 50% material as Citrin® calcium hydroxycitrate taken 3 times daily before meals) given for 12 weeks led to significant weight loss despite no significant change in food intake. The findings were 3.7±3.1 kg active versus 2.4±2.9 kg placebo. Over a 3 month period, these results of less than a pound of additional weight loss per month are hardly impressive; however, the difference is significant (Mattes R D, Bormann L. Effects of (−)-hydroxycitric acid on appetitive variables. Physiol Behav. 2000 Oct. 1-15;71(1-2):87-94). Clouatre and coworkers, as noted already, have demonstrated elsewhere that calcium hydroxycitrate is not well absorbed, yet the longer time frame in this study allowed for a metabolic effect despite no significant anorectc effect. Animal trials using very high dosages of HCA have shown an elevation in energy expenditure (Achmadi S S. The potency of potassium hydroxycitrate derived from gelugur fruit (Garcinia atroviridis) in reducing body weight and cholesterol levels in rats. Hayati (Indonesia) 2001;8(1):23-26; see also Greenwood M R, Cleary M P, Gruen R, Blase D, Stem J S, Triscari J, Sullivan A C. Effect of (−)-hydroxycitrate on development of obesity in the Zucker obese rat. Am J Physiol. 1981 January;240(1):E72-8.).

The clinical trials cited above are evidence that the anorectic effects of HCA should be considered as being separable from its weight loss effects in humans just as animal trials indicate that this is the case in other spedes.

The use of controlled delivery techniques with HCA to bypass release into the stomach is known. Indeed, Clouatre and coworkers employed controlled delivery in a pertinent example described in U.S. Pat. No. 6,207,714 covering the use of HCA for blood glucose and insulin metabolism. At that time and as a result of these experiments, it was shown that the release into the small intestine, although it could have a profound effect on blood sugar, had only a small impact on appetite. This confirmed the hypothesis that increasing blood levels of HCA via enteric delivery so as to potentiate the many metabolic benefits of the compound could be at least partially divorced from the appetite suppressing actions of the substance.

The inventors have explored the interaction of HCA with a number of other compounds. In a pilot study, it was observed that the consumption of hot peppers, for instance, can nullify the immediate anorectic actions of HCA. These results were in-line with published studies demonstrating that capsaicin increases the rate of gastric emptying. (Debreceni A, Abdel-Salam O M, Figler M, Juricskay I, Szolcsanyi J, Mozsik G. Capsaicin increases gastric emptying rate in healthy human subjects measured by ¹³C-labeled octanoic acid breath test. J Physiol Paris. 1999 November;93(5):455-60).

As noted above, HCA is protective against the ulcerative actions of alcohol and indomethacin. Experimentally, it has been shown that capsalcin-sensitive sensory nerves are involved in ulcerations from these sources and that pretreatment with capsaicin attenuates the gastric protection afforded by, for example, the oleanolic acid oligoglycoside momordin Ic. (Matsuda H, Li Y, Yoshikawa M. Roles of capsaicin-sensitive sensory nerves, endogenous nitric oxide, sulfhydryls, and prostaglandins in gastroprotection by momordin Ic, an oleanolic acid oligoglycoside, on ethanol-induced gastric mucosal lesions in rats. Life Sci. 1999;65(2):PL27-32). A link is thus established between the gastro-protective properties of HCA and the gastric motility inhibiting property of the compound. Quite obviously, the dosage prescriptions of Roche and the use of HCA in weight loss have no bearing here.

The explanations for the satiation induced by HCA championed by Sullivan and coworkers is not borne out by recent findings. Direct experimentation in rats has shown that hepatic vagal afferents probably are not involved, albeit gastric branch vagal afferents may be implicated. (Kaplan J M, Siemers W H, Smedh U, Schwartz G J, Grill H J. Gastric branch vagotomy and gastric emptying during and after intragastric infusion of glucose. Am J Physiol. 1997 November;273(5 Pt 2):R1786-92). Clinical trials have shown the HCA can induce a quite massive reduction in food intake with only a minor trend in change in body weight or, vice versa, no significant reduction food intake, yet a significant loss of weight. The inventor's own tests have shown that the release point for HCA, i.e., whether stomach or intestine, is a determining factor in these results. It was hypothesized by the inventors that the appropriate delivery method would induce the feeling of fullness in the stomach at one sitting without any requirement of carbohydrate preloading and without resort to massive doses of HCA. One implication of this knowledge was that HCA can be used for the treatment of conditions related to gastric emptying, but unrelated to weight loss.

U.S. Pat. No. 6,476,071 disclosed that HCA lowers leptin levels. This result subsequently has been confirmed by others and has led on group of researchers to refer to a “leptin-like” effect with HCA. This may be of relevance in light of contemporary research into gastric emptying. Cholecystokinin (CCK) is a major gastrointestinal neuropeptide that is secreted in response to food ingestion. It is involved in the feedback regulation of gastric emptying and also modulates food intake. Leptin, a hormone that regulates food intake and energy balance, is secreted from adipose tissue, gastric mucosa, fundic glands, and other tissues. The gastric effects of leptin activate the brain stem nucleus tractus solitarius (NTS) neurons that respond to gastric vagal stimulation. The distal stomach containing the pylorus determined CCK gastric activity, whereas both the proximal and distal stomach are important for leptin's effect. (Yuan C S, Attele A S, Dey L, Xie J T. Gastric effects of cholecystokinin and its interaction with leptin on brainstem neuronal activity in neonatal rats. J Pharmacol Exp Ther. 2000 October;295(1):177-82). In light of the inventors' own experiments involving HCA and the loss of its satiety effect with the ingestion of hot peppers, it is supportive to find in the literature work on the existence of a functional synergistic interaction between leptin and CCK leading to early suppression of food intake involving CCK-A receptors and capsaicin-sensitive afferent fibers. (Barrachina M D, Martinez V, Wang L, Wei J Y, Tache Y. Synergistic interaction between leptin and cholecystokinin to reduce short-term food intake in lean mice. Proc Natl Acad Sci USA. 1997 Sep. 16;94(19):10455-60). As can be seen, research indicates that receptors controlling gastric emptying can be found in the stomach itself. It is probable that HCA acts on one or more sets of these receptors to influence CCK release or receptor activation.

Many gut-produced and released compounds act upon the brain both via vagal afferents and directly. Gastric distention by itself may activate these systems, again, both locally and in the brain. For instance, above it was noted that gastric leptin activates the brain stem nucleus tractus solitarius (NTS) neurons that respond to gastric vagal stimulation. Similarly, a group of neurons in the caudal nucleus of the solitary tract processes preproglucagon to glucagon-like peptides (GLP)-1 and -2, peptides that inhibit food intake when administered intracerebroventricularly. Significantly, gastric distension that may be considered within the physiological range activates GLP-1/2-containing neurons, suggesting some role in normal satiety. (Vrang N, Phifer C B, Corkern M M, Berthoud H R. Gastric distension induces c-Fos in medullary GLP-1/2-containing neurons. Am J Physiol Regul Integr Comp Physiol. 2003 August;285(2):R470-8. Epub 2003 Apr. 24). In turn, despite its effect on gastric emptying, GLP-1 does not lead to postprandial discomfort because, in part, it allows for gastric accommodation (Delgado-Aros S, Vella A, Camilleri M, Low P A, Burton D D, Thomforde G M, Stephens D. Effects of glucagon-like peptide-1 and feeding on gastric volumes in diabetes mellitus with cardio-vagal dysfunction. Neurogastroenterol Motil. 2003 August; 15(4):435-43). Despite its many insulin-related effects found at elevated dosages, research findings suggest a primarily inhibitory function for GLP-1 involving ileal brake mechanisms. (Nauck M A, Niedereichholz U, Ettler R, Holst J J, Orskov C, Ritzel R, Schmiegel W H. Glucagon-like peptide 1 inhibition of gastric emptying outweighs its insulinotropic effects in healthy humans. Am J Physiol. 1997 November;273(5 Pt 1):E981-8). Because HCA delays gastric emptying and increases gastric volume, there is little question but that HCA also causes the release of GLP-1. Something similar might be said of the other incretin, gastric Inhibitory polypeptide (GIP).

An indication that HCA likely does increase GLP-1 comes from a study with the organic acid sodium proplonate which delayed gastric emptying with a pasta meal and increased the levels of GLP-1. (Frost G S, Brynes A E, Dhillo W S, Bloom S R, McBurney M I. The effects of fiber enrichment of pasta and fat content on gastric emptying, GLP-1, glucose, and insulin responses to a meal. Eur J Clin Nutr. 2003 February;57(2):293-8). Furthermore, whereas Sullivan and coworkers focused on the putative role of the liver in the satiety associated with HCA, the inventors emphasize the fact that de novo lipogenesis also occurs in tissues of the small intestine. This fact is generally overlooked and suggests, as was pointed out above, that just as there are early sensing glucoreceptors in the duodenal mucosa which activate GLP-1 upon saturation with glucose and certain other sugars, one might expect that the presence of HCA to lead to the release of GLP-1 inasmuch as HCA to the cellular machinery looks like the citrate that is generated from excess glucose.

In the end, it is also promising to return to CCK. Studies in humans have repeatedly shown that CCK inhibits food intake. However, a gastric preload is generally necessary to achieve a satiating effect with CCK. Thus, CCK given at physiologically relevant concentrations to fasting humans had no effect on satiety or food intake, while the same infusion rate after a banana preload decreased food intake. (Hellstrom P M, Naslund E. Interactions between gastric emptying and satiety, with special reference to glucagon-like peptide-1. Physiol Behav. 2001 November-December;74(4-5):735-41). This pattern appears to describe the actions of HCA quite well. The compound does not inhibit food intake by itself on an empty stomach, but rather requires food to work. Hence, the inventors argue that HCA acts in part upon CCK receptors in line with recent research findings that the requirement for a negative charge at the CCK-A receptor provided in the natural substrate by a sulfate group can be satisfied by organic acids. (Tilley J W, Danho W, Lovey K, Wagner R, Swistok J, Makofske R, Michalewsky J, Triscari J, Nelson D, Weatherford S. Carboxylic acids and tetrazoles as isosteric replacements for sulfate in cholecystokinin analogues. J Med Chem. 1991 March,34(3):1125-36). CCK acts upon receptors in the stomach, but it is known, as well, to act upon duodenal mucosal receptors which, as noted earlier with HCA, feed to afferents that are sensitive to capsaicin. Research supports the notion that acid inhibitors of gastric emptying are not influenced by serotonin blockade and are enhanced by the presence of sugars.

It is possible to enhance the gastric inhibitory effects of HCA through a variety of means, especially if the compound can be given as part of a foodstuff. Citric acid, sodium citrate and other related compounds should further contribute to inhibiting gastric emptying. (Shiotani A, Saeed A, Yamaoka Y. Osato M S, Klein P D, Graham D Y. Citric acid-enhanced Helicobacter pylori urease activity in vivo is unrelated to gastric emptying. Aliment Pharmacol Ther. 2001 November;15(11):1763-7). In general, lowering pH has a systematic effect in delaying the onset of gastric emptying and increasing gastric residence time (Chaw C S, Yazaki E, Evans D F. The effect of pH change on the gastric emptying of liquids measured by electrical impedance tomography and pH-sensitive radiotelemetry capsule. Int J. Pharm. 2001 Oct. 4;227(1-2):167-75). Similar actions can be expected from sodium propionate, propionic acid, gallic acid and propyl gallate. As discussed above in regard to one study employing HCA, delaying gastric emptying with these organic acids does not necessarily lead to weight loss. In comparative trials using HCA and citrate, the citrate did not have a significant impact upon weight.

A number of plant compounds and extracts have shown the ability to inhibit gastric emptying. These include extracts of marigold (Calendula officinalis), escins and other compounds from Aesculus hippocastanum seeds, extracts of the fruit of Kochia scoparia, and the roots and other parts of Aralia elata, proteinase inhibitor extracts from potato and soybean sources, and a variety of oleanolic acid glycosides from many sources. Other putative delayers of gastric emptying include herbal combinations such as one consisting of yerba mate, damiana and guarana.

It was not known in the art that HCA was useful to delay gastric emptying, that it influenced glucagon-like peptides (GLP-1/2) nor that it influenced cholecystokinin (CCK). Indeed, the primary researchers repeatedly argued that only an indirect mechanism based upon the liver is involved. The present inventors are the first to recognize not only that HCA delays gastric emptying, but also that this allows for the introduction of quite new dosage schedules and the use of HCA in novel areas unrelated to weight loss, such as forms of hypertension, liver dysfunction, and so forth and so on.

HCA-Containing Compounds of the Invention

HCA-containing compounds of the invention which include, but not limited to, e.g., HCA free acid, HCA salts, HCA derivatives, or any combination thereof, to make a granulate which can be used alone or further formulated with pharmaceutically acceptable compounds, vehicles, or adjuvants with a favorable delivery profile, i.e., suitable for delivery to a subject. The free acid form and various salts of (−)-hydroxycitric acid (calcium, magnesium, potassium, sodium and mixtures of these) have been available commercially for several years. Any of these materials can be used to fulfill the invention revealed here, but with varying degrees of success. These materials are generally useful in this descending order of efficacy: potassium salt, sodium salt, free acid, magnesium salt, calcium salt. The previously patented hydroxycitric acid derivatives (mostly amides and esters of hydroxycitric acid, the patents for which are now expired) likely are roughly equivalent to the HCA sodium salt in efficacy.

Such compositions typically comprise the HCA-containing compound of the invention and a pharmaceutically acceptable carrier. As used herein, “pharmaceutically acceptable carrier” is intended to include any and all solvents, dispersion media, coatings, antibacterial and antifungal compounds, isotonic and the like, compatible with pharmaceutical administration. Suitable carriers are described in the most recent edition of Remington's Pharmaceutical Sciences, a standard reference text in the field, which is incorporated herein by reference. A pharmaceutical composition of the invention is formulated to be compatible with its intended route of administration. Examples of routes of administration include parenteral, e.g., intravenous, intradermal, subcutaneous, oral, transdermal (i.e., topical), transmucosal, and rectal administration. The pH can be adjusted with acids or bases, such as hydrochloric acid or sodium hydroxide.

Oral compositions generally include an inert diluent or an edible carrier. They can be enclosed in gelatin capsules, caplets or compressed into tablets. For the purpose of oral therapeutic administration, the HCA-containing compound of the invention can be incorporated with excipients and used in the form of tablets, troches, or capsules. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the HCA-containing compound and the particular therapeutic effect to be achieved, and the limitations inherent in the art of compounding such an active compound for the treatment of individuals.

The discovery that the stomach and the duodenum are the primary sites of action of HCA in delaying gastric emptying is of great importance. Also significant is the fact that the delivery of HCA after the meal, which is to say after the stomach has already begun to empty, is non-productive in this regard. Yet another factor that needs to be taken into the account is the cooperation between HCA and sugars (digestible and many non-digestible, e.g., xylitol) and similar compounds in gastric signalling. Finally, it must be kept clearly in mind that capsaicin and substances similar to capsaicin in their effects upon gastric vagal afferents and other capsaicin-sensitive afferents will nullify the potential of HCA for delaying gastric emptying.

Desirable deliveries must take into account that HCA binds to many gums, fibers, anthocyanins, catechins and other compounds. Color changes in tea and gape juice when salts of HCA are added are immediately visible signals indicating that unwanted changes that are taking place. Insoluble salts, such as calcium HCA, when delivered as tablets or even as capsules may not fully dissolve early enough in the stomach to be efficacious. Calcium makes HCA less active even when present merely as a component fraction of a potassium salt and used to make the potassium salt less hygroscopic (one of the so-called double metal salts). It may be that calcium blocks a potassium-dependent transport channel or otherwise interferes with the impact of HCA upon gastric emptying or even interferes with the metabolic effects of HCA when included as part of the salt. The free acid, similarly, is hard to work with because it lactonizes readily and the lactone is much less active than is the acid.

Formulation and Use of HCA-Containing Compounds to Affect Gastric Emptying and Receptive Relaxation

As detailed above, the literature teaches that HCA compound reduces blood lipids, induces weight loss and decreases appetite in both animals and humans. However, the inventors have discovered that food and pharmaceutical compositions containing (−)-hydroxycitric acid, its salts, amides and esters can be employed for delaying gastric emptying and increasing receptive relaxation for preventing and treating diverse conditions. There are concomitant influences on glucagon-like peptides (GLP-1/2) and cholecystokinin (CCK). Altered gastric emptying and accommodation are found with forms of hypertension, liver dysfunction and gastrointestinal ulcers, especially duodenal ulcer. Numerous medications, such as antibiotics (erythromycin, indomethacin, etc). and including even some diet drugs (e.g., Orlistat and other lipase inhibitors), can accelerate gastric emptying. Surgery, such as for peptic ulcers, itself can lead to clinical dumping syndrome, as can other types of surgery performed on the stomach. Other factors or conditions that lead to acceleration of gastric emptying include obesity, high-energy density of food, fat intolerance, early stages of noninsulin-dependent diabetes mellitus, Zollinger-Ellison syndrome, and intermittent episodes in other forms of diabetes. HCA delivered in the form of its potassium salt is efficacious at singly delivered dosages of between 150 mg and 5 grams, preferably at a dosage of between 500 mg and 3 grams for most individuals. Other salts, amides and esters are active at individual dosage ranges, with, for instance, the sodium salt acting similarly to the potassium salt whereas salts containing calcium are less active. Various delivery methods that preferentially expose HCA to stomach and duodenal receptors and sensors without undue binding of the compound to inactivating substances are provided. The safe and effective employment to delay gastric emptying is an entirely novel use of (−)-hydroxycitric acid, its derivatives and its salt forms.

Methods for taking advantage of the present invention include, but are not limited to the following in addition to one or more sources of HCA. These items are intended to provide for “instant release” into the stomach, be released by chewing or upon exposure to stomach acid, and so forth. Employment of the salts of HCA that are most active in producing satiety (potassium and sodium) requires the concomitant application of one or more of the delivery methods (patented and patent-pending) developed by the inventors to render these hygroscopic salts workable. Examples given below elaborate and extend methods for

1) capsules or tablets containing sodium bicarbonate, potassium bicarbonate or (less advantageously) calcium carbonate sufficient to cause the rapid release of the contents of the capsule or tablet when exposed to stomach contents

2) capsules or tablets containing sodium bicarbonate or potassium bicarbonate plus alginic acid; also capsules or tables containing sodium or potassium alginate to achieve a prolonged dwell time in the stomach and extended presentation to the stomach wall

3) dry packaged powders designed to mixed with water or juice and consumed between meals or

prior to meals; HCA mixed into tomato juice is especially successful, whereas HCA tends to
bind to components in citrus juices; precoating of the HCA with hydrophobic components is necessary before placing the salts in packaged materials
   4) special processing of HCA salts, etc., for instance, with molten oils such as hydrogenated vegetable oil, glycerol monosterate, cetyl alcohol, stearyl alcohol and various high viscosity grades of conjugated polyethylene glycol, d-α-tocopheryl polyethylene glycol succinate (TPGS) and similar compounds (see inventors' U.S. patent application Ser. No. 10/447,992), after which this material, now rendered non-hygroscopic and non-reactive, is further encased in gelatin, tapioca, gums/pectins, inulin, cellulose derivatives, etc., for inclusion in thick drinks, soft-center bars and candies, pudding snacks, jelly-like confections, “gummy” deliveries, liquid meal replacements, etc.
   5) the inclusion with or use in conjunction with HCA of other agents that influence gastric emptying, such as citric acid, sodium or potassium citrate, other citric acid salts, sodium propionate, propionic acid, gallic acid, propyl gallate; extracts of marigold (Calendula officinalis); escins and other compounds from Aesculus hippocastanum seeds; extracts of the fruit of Kochia scoparia, and the roots and other parts of Aralia elata; saponins, especially theasaponin E1 from the seeds of the tea plant (Camellia sinensis L).; extracts from bay leaf (Laurus nobilis), especially costunolide and its active component, alpha-methylene gamma-butyrolactone (alpha-MGBL); proteinase inhibitor extracts from potato and soybean sources; a variety of oleanolic acid glycosides from many sources; also herbal combinations such as one consisting of yerba mate, damiana and guarana.

The following are examples of supporting data and means of application for the invention.

### EXAMPLE 1

### Human Trial of HCA-Containing Compound of the Invention

Evidence that HCA during the initial weeks of usage likely reduces appetite through an effect upon gastric emptying emerged from a clinical trial of an immediate-release formulation of HCA-containing compound of the invetion. Previously, Sullivan and coworkers, in public documents, had shown that HCA can control food intake if administered in one large bolus dose or in two much smaller dosages given prior to meals. This can be interpreted either as indicating the clearance rate of the drug or as indicating a mechanism. HCA given after a meal has already begun has no impact upon food intake; the stomach must become again completely empty before anorexia returns. However, HCA given continuously in the food supply to rats, animals which eat more or less continuously during waking hours if food is available will, again, reduce food intake. Sullivan and coworkers argued in numerous public documents that the appetite suppressing actions of HCA depend upon the activation of glucoreceptors in the liver, yet this particular explanation for a peripherally-acting agent (no effect upon the central nervous system) seems inadequate in light of the very quick onset of satiety after a meal has begun in experiments in which animals are restricted to two meals per day after gavage with the compound. It also seems to be inadequate given that in an experiment in which the rats' satiety center of the brain had been destroyed there still was appetite suppression. Hepatic glucoreceptor activation of the vagus nerve probably would have no impact upon the satiety center of the brain under such circumstances. Hence, meals dearly trigger some mechanism which has been activated by HCA. Moreover, it is unlikely that sufficient calories from a meal can reach the liver in time to account for the rapid onset of satiety or satisfy these other conditions just mentioned. In a drug which acts at least in part upon receptors in the stomach and/or small intestine, these factors, however, would no longer be problematic.

In the present studies, data on human usage emerged from a multi-week pilot open clinical weight loss trial with extremely obese patients which was undertaken to gauge the effects of a pouch delivery form of a potassium salt of HCA under the normal circumstances faced in clinical practice with this patient population. Sixteen patients were enrolled, three of whom were diabetics on medications and several others who were suspected of suffering from insulin resistance. The patients ingested 3-4 grams of HCA (in the form of the potassium salt) per day in two divided doses. Aside from being informed that they must eat a carbohydrate-containing meal within one hour of taking the HCA and that they should avoid eating late in the day, they were not instructed to follow any special diet or exercise plan outside their normal habits and no caloric restriction was imposed. This particular form of potassium HCA delivery typically was mixed into water or juice and consumed at mid-morning and mid-afternoon. The delivery was a water-soluble immediate release form. It was a pre-commercial preparation and nearly all of the patients complained regarding the inconvenience and poor taste of the product, albeit there were no other issues of tolerability.

A number of patients continued on the program for 6 weeks. However, most patient data was good for only 3 weeks because two of the diagnosed diabetics experienced severe hypoglycemic reactions. Several other patients experienced good appetite suppression, yet also complained of episodic tiredness at the beginning of the program, a sign of low blood sugar. Two patients subsequently were placed on phentermine. One patient who followed the program for 10 weeks with excellent weight loss (32 pounds over 10 weeks) found that his tendency toward elevated blood sugar was stabilized during the program. This patient returned to his prior experiences of infrequent hypoglycemia roughly one week after he had left the program, something which suggests a carryover effect from the compound. The average weight loss over the 3 week period for these 14 patients was approximately 3.1 pounds per person per week. In the eight patients with hypertension, the compound showed a surprising ability to normalize blood pressure. The clinical decision was made that potassium HCA in an immediate release format can exercise a strong hypoglycemic effect in diabetics and that it appears to influence blood sugar levels in protodiabetics, as well. At therapeutically effective dosages, HCA probably should be used with diabetic populations only under a physician's care.

When questioned regarding degree of appetite suppression and compliance patterns in taking the HCA, many patients noted that not only did the compound make them “feel fuller faster,” but also that they seemed to feel full for a longer period of time. The authors speculated that rapidity of onset of satiety may involve intestinal glucorecptors and that continued satiety could involve these same receptors or some allied mechanism. For instance, protease inhibitors which block trypsin and chymotrypsin may enhance satiety by preventing digestion of the cholecystokinin-releasing peptide (CCK-RP), a peptide which is secreted into the gut lumen during meals. CCK-RP can then stimulate release of the satiety peptide CCK from endocrine cells in the small intestine.

### EXAMPLE 2

### Metabolic Effect with out Appetite Control

In Example 1, the HCA was delivered in an immediate-release preparation. Our unexpected findings with regard to blood sugar led to the hypothesis that a relatively large dose of HCA might affect blood sugar levels in an individual whose blood sugar is in the low normal range. A dose of 1.5 grams HCA derived from potassium HCA and delivered in a special coated form designed to bypass interaction with stomach acids and to release only in the higher pH of the small intestine was used. A potassium HCA salt granulate was prepared according to Example 1 found in U.S. Pat. No. 6,447,807 and delivered via a dry powdered meal replacement designed to be mixed with liquid to provide 1.5 grams of HCA per 350 calories plain (mixed with water) or approximately 500 calories with milk. After an overnight fast, the subject had a measured blood glucose level of 85 mg/dL. The subject ate a 500 calorie breakfast consisting the experimental HCA meal replacement. Two hours after this meal, subject's blood glucose level had dropped to 77 mg/dL. The subject reported no changes in energy levels, but this subject was known to metabolize fats well as fuel, hence was not expected to experience low energy. Striking at the time was the fact that delivery of potassium HCA to the small intestine and by-passing the stomach appeared to blunt the anorectic actions of the drug. This finding seemed paradoxical in that the outstanding metabolic effect, which might be thought to indicate blood levels of the drug, was not matched by even a normal level of feelings of fullness. This implied that at least part of the satiation induced by HCA comes about prior to entry of the compound into the blood. As noted in the text, studies published subsequent to our own research have shown the same pattern of at least partial disconnect between metabolic and appetite effects of HCA. (Westerterp-Plantenga M S, Kovacs E M. The effect of (−)-hydroxycitrate on energy intake and satiety in overweight humans. Int J Obes Relat Metab Disord. 2002 June;26(6):870-2; Mattes R D, Bormann L. Effects of (−)-hydroxycitric acid on appetitive variables. Physiol Behav. 2000 Oct. 1-15;71(1-2):87-94.)

### EXAMPLE 3

### Leptin, a Known Link to Cholecystokinin (CCK)

Very recently, Japanese researchers gave HCA to mice on a 10% sucrose diet and observed that levels of serum insulin and leptin as well as the leptin/white adipose tissue ratio were lower in the treated mice than in the control. They concluded that “these findings suggested that G. cambogia extract efficiently improved glucose metabolism and displayed leptin-like activity.” (Hayamizu et al., Fitoterapia. 2003 April;74(3):267-73). The gastric effects of leptin activate the brain stem nucleus tractus solitarius (NTS) neurons that respond to gastric vagal stimulation. The distal stomach containing the pylorus determines CCK gastric activity, whereas both the proximal and distal stomach are important for leptin's effect. (Yuan et al., J Pharmacol Exp Ther. 2000 October;295(1):177-82). Various researchers have demonstrated an interaction between leptin and cholecystokinin. (Barrachina et al., Proc Natl Acad Sci USA. 1997 Sep. 16;94(19):10455-60).

U.S. Pat. No. 6,476,071 disclosed that HCA alters insulin and leptin levels. When considered in the light of other evidence regarding the appetite suppression found with HCA, such findings provide reasonable evidence that HCA likely activates or interacts synergistically with CCK. In one study mentioned in our earlier patent, the inventors arranged for male OM rats aged 10 weeks to be fed a diet in which 30% of the calories were obtained from fat under standard conditions (U.S. Pat. No. 6,476,071).

The rats were intubated twice daily with one of three HCA salts or placebo. The amount of HCA in each arm of 5 animals was the minimum dosage which had been found effective in the form of the pure trisodium salt of HCA in tests by Hoffmann-La Roche (see Sullivan et al., supra) in animals ingesting a 70% glucose diet, i.e., 0.33 mmoles/kg body weight HCA given twice per day. The HCA salts used were these: CaKHCA=a mixed calcium and potassium HCA salt commercially marketed as being entirely water soluble; KHCA 1=a relatively clean, but still hardly pure potassium salt of HCA with a good mineral ligand attachment supplying 4467 mg potassium/100 grams of material; KHCA 2=an impure potassium salt of HCA with large amounts of gums attached and poor mineral ligand attachment supplying 2169 mg potassium/100 grams of material. Data was collected from the rat study with regard to serum insulin, leptin and corticosterone levels and is summerized below in Table 1.

TABLE 1

| Group | Insulin ng/mL | Leptin ng/mL | Corticosterone ng/mL |
|---|---|---|---|
| Control | 2.655 | 9.52 | 269.38 |
| Control | 7.077 | 18.94 | 497.87 |
| Control | 4.280 | 34.34 | 265.71 |
| Control | 9.425 | 24.32 | 209.54 |
| Control | 3.798 | 8.40 | 116.12 |
| KHCA 1 | 3.880 | 9.93 | 45.79 |
| KHCA 1 | 4.399 | 7.31 | 33.10 |
| KHCA 1 | 3.181 | 9.25 | 65.57 |
| KHCA 1 | 3.210 | 24.36 | 55.40 |
| KHCA 1 | 3.639 | 9.07 | 84.62 |
| KHCA 2 | 4.427 | 9.13 | 26.02 |
| KHCA 2 | 4.301 | 9.75 | 270.83 |
| KHCA 2 | 3.245 | 8.00 | 45.44 |
| KHCA 2 | 3.695 | 9.16 | 45.63 |
| KHCA 2 | 2.053 | 8.26 | 38.04 |

Both of the potassium HCA arms were superior to the calcium/potassium arm (data not shown) in reducing insulin, leptin and corticosterone concentrations. Because of the difficulty in achieving significance with only 5 data points per arm, calculations regarding insulin and leptin combined the data from the two KHCA arms. With respect to insulin, the one-tailed P value was a significant 0.0306, and the two-tailed P value fell slightly short of significance at 0.0612. Using this combined data, there was also a significant one-tailed P value difference between the two KHCA arms and the result found with the CaKHCA. With respect to leptin, the two KHCA arms were combined, in part, because of one anomalously high data point and yielded a one-tailed P value which was a significant 0.0241 and a two-tailed P value which was significant at 0.0482. Corticosterone results were highly significant even at 5 data points per arm. KHCA 1 was easily significantly superior to control: the one-tailed P value was a highly significant 0.0048, and the two-tailed P value was a highly significant 0.0096.

The implication of these data is that HCA, if supplied in appropriate amounts, may be useful in reducing insulin levels and insulin resistance, leptin levels and leptin resistance, and elevated glucocorticoid levels. Therefore, the inventors' data supports a conclusion that HCA displays “leptin-like” activity. Moreover, the effect of HCA on leptin levels was significantly stronger with KHCA than with the double-metal calcium and potassium salt. This disparity was paralleled by the greater appetite/food intake and weight gain found with the double-metal calcium and potassium salt which, on the high-fat diet employed in this study, led to food intake and weight gain greater than that found in control. Hence, we have indirect evidence from our own study of a link between the ingestion of HCA and the regulation of components known to interact with leptin, in this case CCK. It is not yet known why or how calcium interferes with the actions of HCA when used as a cation.

### EXAMPLE 4

### Capsaicin Defeats HCA-Induced Satiety

The research literature supports a functional synergistic interaction between leptin and CCK leading to early suppression of food intake involving CCK-A receptors and capsaicin-sensitive afferent fibers. (Barrachina et al., Proc Natl Acad Sci USA. 1997 Sep. 16;94(19):10455-60). This research indicates that receptors controlling gastric emptying can be found in the stomach itself. Other work demonstrates that capsaicin increases the rate of gastric emptying (Debreceni et al., J Physiol Paris. 1999 November;93(5):455-60).

To test whether there is a capsaicin-HCA interaction as is suggested by our proposed effect upon CCK, the inventors invited 5 individuals to consume approximately 2 grams potassium HCA in water about an hour before a meal. The meal itself began with a soup course. The participants reported that they felt full very soon after beginning to consume the likewise savory, but non-spicy main portion of the meal. At this point, bottles of red pepper sauce were supplied and the sauce was applied liberally. Shortly thereafter, the participants found that they could “eat through” the previous feeling of fullness. As is true of Example 3, this provides indirect evidence that HCA acts upon a CCK-related mechanism in inducing satiety.

### EXAMPLE 5

### Immediacy of HCA-Induced Satiety

Contrary to the conclusions in the scientific literature based upon rats studies, the inventors postulated that HCA's satiety is related to the volume of stomach contents rather than to the number of calories that have been presented to the liver. It is known that glucagon-like peptide has two points of action; the first occurs almost immediately as food begins to be ingested and influences gastric emptying, whereas the second occurs only much later and influences the tenacity of the satiety. Again, the first action of GLP-1 may in part be in response to gastric extension and may lead to both direct and vagally-mediated effects in the brain. A gastric preload also is generally necessary to achieve a satiating effect with CCK. Thus, CCK given at physiologically relevant concentrations to fasting humans had no effect on satiety or food intake, while the same infusion rate after a banana preload decreased food intake. (Hellstrom and Naslund, Physiol Behav. 2001 November-December;74(4-5):735-41). In other words, gastric volume and the act of loading the stomach seem to be important both for the first mechanism associated with GLP-1 and for the anorectic effect of CCK.

The inventors reasoned that if HCA quickly intervenes to delay gastric emptying and the mechanisms involved do not involve glucose receptors in the liver, then even consuming a drink characterized by high volume, but relatively few calories might lead to satiety. To test this theory, and the palatability of an HCA salt when mixed with various flavors, a study was conducted in which 5 individuals consumed approximately 2 grams potassium HCA mixed in sweetened lemonade-like drinks prior to a meal. Consumption of the drinks took place over the course of approximately one half hour and involved 16-24 ounces of fluid, but only about 200 calories. As is well established, beverages do not normally have great satiating power. Nevertheless, all the participants found that they were satiated soon after the meal began. This example strongly suggested that gastric emptying and quick-acting satiety mechanisms are brought into play by HCA.

### EXAMPLE 6

### Fast-Acting Capsule and Tablet Composition

All of the standard salts of HCA can be delivered after a fashion that rapidly increases exposure to the stomach lumen through the use of capsules or tablets containing sodium bicarbonate, potassium bicarbonate, magnesium carbonate or (less advantageously) calcium carbonate and similar compounds sufficient to cause the rapid release of the contents of the capsule or tablet when exposed to stomach contents. Hygroscopic salts of HCA, such as the potassium and sodium salts, will require initial processing with hydrophobic (but not acidophobic) coatings, etc. before being added to the capsules or tablets.

In one embodiment of the invention, an HCA-containing composition useful to delay gastric emptying in a subject is the composition detailed below in Table 2.

TABLE 2
Example of a Fast-Releasing Formulation

| Product | Mg/Capsule | % |
|---|---|---|
| 1. Potassium-calcium HCA | 200 mg | 50.0 |
| 2. Sodium Bicarbonate | 30 mg | 7.50 |
| 3. Starch 1500 | 70 mg | 17.50 |
| 4. Malic Acid | 100 mg | 25.00 |
| TOTAL | 400 mg | 100.0% |

The HCA salt was blended with starch 1500 and sodium bicarbonate; malic acid was then added and blended and the whole powered material was passed through a #20 screen to allow even pouring and filling of capsules. If it was desired to make tablets out of this material, it was mixed with 0.5% magnesium stearate and compressed on a rotary tablet machine. After entering the stomach the starch initiated the immediate disintegration of the tablet or capsule and the sodium bicarbonate mixed with the malic acid to cause the rapid dispersal of the HCA. Numerous additional acids can be used to activate the bicarbonate, such as L-tartaric acid, citric acid, lactic acid, alginic acid, fumaric acid, aspartic acid and ascorbic acid. The formula can also omit the acid component and depend entirely upon the gastric acid of the stomach to induce the reaction with the bicarbonate.

### EXAMPLE 7

### Sustained Gastric Residence Compostion of the Invention

All of the standard salts of HCA can be delivered after a fashion that increases mean residence time in the stomach extended presentation to the stomach wall through the use capsules or tablets containing sodium bicarbonate or potassium bicarbonate plus alginic acid; also capsules or tables containing sodium or potassium alginate. Hygroscopic salts of HCA, such as the potassium and sodium salts, will require initial processing with hydrophobic (but not acidophobic) coatings, etc. before being added to the capsules and tablets.

One means of increasing the residence time of HCA in the stomach is to use the simple formula in Example 6 and substitute alginic acid for malic acid. Sustaining the residence time of the HCA in the stomach also can be accomplished by using an aqueous latex dispersion of ethyl cellulose known commercially as Surerelease® or Aquatcoat®. This can be sprayed onto the non- and moderately-hygroscopic HCA salts, such as the calcium and potassium-calcium salts, in a fluid bed dryer in a 0.5-1% coat. (Fully hygroscopic salts of HCA, such as the pure potassium and sodium salts, except under very dry conditions may first need to be pre-coated (The hygroscopic nature of pure HCA salts is discussed in Jena B S, Jayaprakasha G K, Singh R P, Sakariah K K. Chemistry and biochemistry of (−)-hydroxycitric acid from Garcinia. J Agric Food Chem. 2002 Jan. 2;50(1):10-22; see also U.S. Pat. No. 6,447,807).

The coated material can then be admixed with alginic acid and sodium bicarbonate along with starch. The light water impermeable coat will dissolve from the HCA before being expelled from the stomach and some will be trapped in the foamy alginate bicarbonate material which will prolong it's dwell time in the stomach. A capsule formulation of one embodiment of the invention, is detailed below in Table 3.

TABLE 3
Sustained Residence Formulation

| Product | Mg/Capsule | % |
|---|---|---|
| 1. Potassium-calcium HCA | 400 mg | 57.14% |
| 2. Sodium Bicarbonate | 50 mg | 7.14% |
| 3. Alginic Acid | 200 mg | 28.58% |
| 4. Starch | 50 mg | 7.14% |
| TOTAL | 700 mg | 100.0% |

The HCA was first sprayed with a latex dispersion of ethyl cellulose. When it is dry, it was blended with the remaining materials and placed through a #20 screen. When this was complete, the milled granulate was placed into capsules with a weight of 700 mg or compressed into tablets of similar weight. The disintegration rate should be 100% within 20 minutes.

### EXAMPLE 8

### Dry Packaged Meal Replacement Composition

It is feasible to supply HCA via dry packaged powders designed to be mixed with water or juice and consumed between meals or prior to meals. HCA mixed into tomato juice was especially successful, whereas HCA tends to bind to components in citrus, grape and many other juices. Under normal commercial processing, sufficient moisture remains in food products to allow even HCA calcium salts to slowly bind to food components, such as tannins, gums, fibers and pectins. The much more active potassium and sodium salts of HCA are not practical unless they have undergone initial processing with hydrophobic coatings.

All of the commercial salts of HCA will bind to food components in dry mixtures if left in contact for any extended length of time. A lack of awareness of the fact that HCA salts must be prevented from being inactivated by food elements, phytonutrients, etc., has contributed greatly to failed and disappointing trials using the compound. Hence pretreatment of some sort is absolutely necessary.

Potassium-calcium HCA can be coated with a small dose of ethyl cellulose such as noted in example 7 and placed in a vacuum sealed envelope after being mixed with dried food and/or herb concentrates. The contents of the package later can be mixed with water and ingested 30 minutes to 1 hour before a regular meal or as a snack before bedtime. Capsaicin-based condiments and flavorings, such as pepper sauces, should be avoided in these snacks and meal replacements.

### EXAMPLE 9

### Compositions of the Invention for Use In Liquids, Bars, Jelly-Like Products, and the Like

Because of the resulting non-gritty mouth feel, it is especially advantageous to pre-treat HCA salts with molten oils such as hydrogenated vegetable oil, glycerol monosterate, cetyl alcohol, stearyl alcohol and various high viscosity grades of conjugated polyethylene glycol, d-α-tocopheryl, polyethylene glycol succinate (TPGS) and similar compounds prior to being added to foodstuffs. Subsequent processing allowed the material, now rendered non-hygroscopic and non-reactive, to be further encased in gelatin, tapioca, gums/pectins, inulin, cellulose derivatives, etc., for inclusion in thick drinks, soft-center bars and candies, pudding snacks, jelly-like confections, “gummy” deliveries, liquid meal replacements, etc. Upon consumption, the HCA is released by mechanical means (chewing) and enters the stomach in conjunction with food and liquid. As such, the dosage of HCA can be taken via snacks or meal replacements and is accompanied by the items necessary to supply the volume that activates HCA-induced satiety.

### EXAMPLE 10

### Compounds for Additive and Synergistic Benefits

HCA may be used in conjunction with many agents that influence gastric emptying, such as citric acid, sodium or potassium citrate, other citric acid salts, sodium propionate, propionic acid, gallic acid, propyl gallate; extracts of marigold (Calendula officinalis); escins and other compounds from Aesculus hippocastanum seeds; extracts of the fruit of Kochia scoparia, and the roots and other parts of Aralia elata; saponins, especially Theasaponin E1 from the seeds of the tea plant (Camellia sinensis L).; extracts from bay leaf (Laurus nobilis), especially costunolide and its active component, alpha-methylene-gamma-butyrolactone (alpha-MGBL); proteinase inhibitor extracts from potato and soybean sources; a variety of oleanolic acid glycosides from many sources; also herbal combinations such as one consisting of yerba mate, damiana and guarana. In one embodiment of the invention, an HCA-containing composition useful to delay gastric emptying in a subject is the composition detailed below in Table 4.

TABLE 4
Example of a Synergistic Fast-Releasing Formulation

| Product | Mg/Capsule |
|---|---|
| 1. Potassium-calcium HCA | 200 mg |
| 2. Sodium Bicarbonate | 30 mg |
| 3. Starch 1500 | 70 mg |
| 4. Malic Acid | 100 mg |
| 5. Yerbe mate | 112 mg |
| 6. Guarana | 95 mg |
| 7. Damiana | 36 mg |
| TOTAL | 643 mg |

The HCA salt was blended with starch 1500 and sodium bicarbonate; malic acid, yerbe maté, guarana and damiana are then added and blended. The resultant whole powered material was passed through a #20 screen to allow even pouring and filling of capsules. If it was desired to make tablets out of this material, it was mixed with 0.5% magnesium stearate and compressed on a rotary tablet machine. Three capsules are taken three times per day 30 to 60 minutes before meals with 8-16 ounces of apple, tomato or other juice; alternatively, 4 or 5 capsules are taken twice per day prior to lunch and supper.

### EXAMPLE 11

### Soft Gelatin Encapsulation of HCA

Soft gelatin encapsulation was used for oral administration of drugs in liquid form. For this purpose, HCA was provided in a liquid form by suspending it in oils, polyethylene glycol-400, other polyethylene glycols, poloxamers, glycol esters, and acetylated monoglycerides of various molecular weights adjusted such as to insure homogeneity of the capsule contents throughout the batch and to insure good flow characteristics of the liquid during encapsulation. The basic ingredients of the shell were gelatin, plasticizer, and water. Care was exercised in the case of softgels to use the less hygroscopic salts and forms of HCA or to pretreat the more hygroscopic salts to reduce this characteristic. The carrier was adjusted depending on the HCA salt, ester or amide used so as to avoid binding of the ingredients to the carrier.

### EXAMPLE 12

### Fast-Disintegrating

Very fast disintegrating or “explosive” tablets were formulated to quickly delivery HCA to the receptors in the upper gastrointestinal tract. These tablets exhibited >90% dissolution within 3-4 minutes when agitated in a pH ˜2.0. The product began with a granulate prepared as follows:

HCActive 60% HCA Granulation using 2% Water-soluble
Kollicoat IR Coating

| | |
|---|---|
| HCA potassium/magnesium salt | 2.000 kg |
| Kollicoat IR | 0.040 kg |
| Water | 0.380 kg |
| Yield: | 2.040 kg |

Coating Technique Information for Fluid Bed Dryer

Spray Rate: 9%
Outlet: 25-33° C.
Inlet: 45-55° C.
Atomizer: 55 PSI
CFM: 200-400
Dried to: 45° C. outlet temperature; inlet less than 60° C.

In one embodiment of the invention, an HCA-containing composition useful to delay gastric emptying in a subject is the composition detailed below In Table 5.

TABLE 5

| | Item | | Amount | | |
|---|---|---|---|---|---|
| Item # | Key | Ingredient | (mg/Tablet) | Wt (Kg) | Percent |
| 1 | Premix | HCActive (60%) | 833.33 | 2.714 | 58.1 |
| 2 | | Satialgine | 200 | 0.651 | 13.9 |
| 3 | | Sodium Bicarbonate | 200 | 0.651 | 13.9 |
| 6 | | Citric Acid | 200 | 0.651 | 13.9 |
| 7 | | Magnesium Stearate | 7 | 0.0234 | 0.50 |
| | | TOTAL | 1440.530 | 4.668 | 100.3 |

1. Items #1-7 were weighed and blended.
2. The mixed granulate was then placed on a rotary press and compressed into tablets with a weight of approximately 1440.53 mg and a fracture force of 10-15 kg.

### EXAMPLE 13

### Chewable Preparations of the HCA-Containing Compound of the Present Invention

For the purposes of the invention, various chewable preparations are desirable. Because these are broken down completely in the mouth, they are quite effective in presenting HCA to the sensors in the stomach and duodenum. In this example, orange color and flavor were used, as was Aspartame as a sweetener. However, other flavors, such as chocolate and a chocolate plus peppermint flavor have been used successfully, as has sweetening with stevia powder. The starting material was a precoated HCActive granulate produced as described above.

In one embodiment of the invention, an HCA-containing composition useful to delay gastric emptying in a subject is the composition detailed below in Table 6.

TABLE 6

| | Item | | Amount | Wt | |
|---|---|---|---|---|---|
| Item # | Key | Ingredient | (mg/Tablet) | (Kg) | Percent |
| 1 | Premix | HCActive (60%) | 1,600 | 0.400 | 73.586 |
| 2 | MAN- | Mannitol | 500 | 0.125 | 22.996 |
| | 14 | | | | |
| 3 | | Satialgine | 7 | 0.002 | 0.322 |
| 6 | | Aspartame | 14 | 0.004 | 0.644 |
| 7 | ORA- | Orange color | 53.32 | 0.0133 | 2.452 |
| | 11 | | | | |
| 8 | CUR- | Orange Flavor | 64.00 | 0.0160 | 2.943 |
| | 03 | | | | |
| 9 | MAG- | Magnesium | 13.33 | 0.0033 | 0.613 |
| | 02 | Stearate+UZ,24/32 | | | |
| | | TOTAL | 2251.650 | 0.544 | 1.000 |

1. Items #1-9 were weighed and blended.
2. The mixed granulate was then placed on a rotary press and compressed into tablets with a weight of approximately 2251.65 mg and a fracture force of approximately 10 kg.

### Equivalents

From the foregoing detailed description of the invention, it should be apparent that unique HCA containing compounds and methods of the same have been described resulting in improved HCA containing formulations suitable to affect gastric emptying and increasing receptive relaxation in a subject. Although particular embodiments have been disclosed herein in detail, this has been done by way of example for purposes of illustration only, and is not intended to be limiting with respect to the scope of the appended claims which follow. In particular, it is contemplated by the inventor that substitutions, alterations, and modifications may be made to the invention without departing from the spirit and scope of the invention as defined by the claims. For instance, the choice of HCA salt, encapsulating agent or the choice of appropriate patient therapy based on these is believed to be matter of routine for a person of ordinary skill in the art with knowledge of the embodiments described herein.

What is claimed is:

1. A method for delaying gastric emptying and increasing receptive relaxation in individuals in need thereof which is comprised of administering orally an effective amount of (−)-hydroxycitric acid or one or more pharmaceutically effective and acceptable salts or derivatives of (−)-hydroxycitric acid selected from the group consisting of the free acid or its lactone, the alkali metal salts potassium or sodium HCA, the alkaline earth metal salts calcium or magnesium HCA, a mixture the alkali metal salts and/or the alkaline earth metal salts of HCA or some mixture of alkali metal salts and alkaline earth metal salts of HCA or in the form of therapeutically effective amide and/or ester derivatives of (−)-hydroxycitric acid.

2. A method for influencing glucagon-like peptides in individuals in need thereof which is comprised of administering orally an effective amount of (−)-hydroxycitric acid or one or more pharmaceutically effective and acceptable salts or derivatives of (−)-hydroxycitric acid selected from the group consisting of the free acid or its lactone, the alkali metal salts potassium or sodium HCA, the alkaline earth metal salts calcium or magnesium HCA, a mixture the alkali metal salts and/or the alkaline earth metal salts of HCA or some mixture of alkali metal salts and alkaline earth metal salts of HCA or in the form of therapeutically effective amide and/or ester derivatives of (−)-hydroxycitric acid.

3. A method for influencing cholecystokinin in individuals in need thereof which is comprised of administering orally an effective amount of (−)-hydroxycitric acid or one or more pharmaceutically effective and acceptable salts or derivatives of (−)-hydroxycitric acid selected from the group consisting of the free acid or its lactone, the alkali metal salts potassium or sodium HCA, the alkaline earth metal salts calcium or magnesium HCA, a mixture the alkali metal salts and/or the alkaline earth metal salts of HCA or some mixture of alkali metal salts and alkaline earth metal salts of HCA or in the form of therapeutically effective amide and/or ester derivatives of (−)-hydroxycitric acid.

4. The method of claim 1 where the (−)-hydroxycitric acid is supplied as a therapeutically effective amount as the free acid, its lactone or as one or more of the salts or other derivatives of the free acid and is delivered in a controlled release form.

5. The method of claim 1 where the salts or derivatives are administered orally as a tablet or capsule wherein the contents of said capsule or tablet further comprise sodium bicarbonate, calcium carbonate, or potassium bicarbonate for producing carbon dioxide gas on contact with the stomach liquids wherein the amount of sodium bicarbonate, calcium carbonate or potassium bicarbonate is sufficient to cause the breakup of the capsule or tablet thus releasing the salts or derivatives, but insufficient to cause distension of the stomach.

6. The method of claim 1 where the salts or derivatives are administered orally as a tablet or capsule wherein the contents of said capsule or tablet further comprise sodium bicarbonate or potassium bicarbonate plus alginic acid; also capsules or tables containing sodium or potassium alginate.

7. The method of claim 1 where the salts or derivatives are administered orally as dry packaged powders designed to be mixed with water or juice and consumed between meals or prior to meals.

8. The method of claim 1 where the salts or derivatives are administered orally and are further encased in materials selected from the group consisting of gelatin, tapioca, gums, pectins, inulin, cellulose derivatives, alginic acid, dextran and dextrin for inclusion in thick drinks, soft-center bars and candies, pudding snacks, jelly-like confections, “gummy” deliveries and liquid meal replacements.

9. The method of claim 1 where the salts or derivatives are administered orally in conjunction with materials selected from the group consisting of citric acid, sodium or potassium citrate, other citric acid salts, sodium propionate, propionic acid, gallic acid, propyl gallate; extracts of marigold (Calendula officinalis); escins and other compounds from Aesculus hippocastanum seeds; extracts of the fruit of Kochia scoparia, and the roots and other parts of Aralia elata; saponins, especially Theasaponin E1 from the seeds of the tea plant (Camellia sinensis L).; extracts from bay leaf (Laurus nobilis), especially costunolide and its active component, alpha-methylene-gamma-butyrolactone (alpha-MGBL); proteinase inhibitor extracts from potato and soybean sources; a variety of oleanolic acid glycosides from many sources; also herbal combinations such as one consisting of yerba mate, damiana and guarana.

10. The method of claim 2 where the (−)-hydroxycitric acid is supplied as a therapeutically effective amount as the free acid, its lactone or as one or more of the salts or other derivatives of the free acid and is delivered in a controlled release form.

11. The method of claim 2 where the salts or derivatives are administered orally as a tablet or capsule wherein the contents of said capsule or tablet further comprise sodium bicarbonate, calcium carbonate, or potassium bicarbonate for producing carbon dioxide gas on contact with the stomach liquids wherein the amount of sodium bicarbonate, calcium carbonate or potassium bicarbonate is sufficient to cause the breakup of the capsule or tablet thus releasing the salts or derivatives, but insufficient to cause distension of the stomach.

12. The method of claim 2 where the salts or derivatives are administered orally as a tablet or capsule wherein the contents of said capsule or tablet further comprise sodium bicarbonate or potassium bicarbonate plus alginic acid; also capsules or tables containing sodium or potassium alginate.

13. The method of claim 2 where the salts or derivatives are administered orally as dry packaged powders designed to be mixed with water or juice and consumed between meals or prior to meals.

14. The method of claim 2 where the salts or derivatives are administered orally and are further encased in materials selected from the group consisting of gelatin, tapioca, gums, pectins, inulin, cellulose derivatives, alginic acid, dextran and dextrin for inclusion in thick drinks, soft-center bars and candies, pudding snacks, jelly-like confections, “gummy” deliveries and liquid meal replacements.

15. The method of claim 2 where the salts or derivatives are administered orally in conjunction with materials selected from the group consisting of citric acid, sodium or potassium citrate, other citric acid salts, sodium propionate, propionic acid, gallic acid, propyl gallate; extracts of marigold (Calendula officinalis); escins and other compounds from Aesculus hippocastanum seeds; extracts of the fruit of Kochia scoparia, and the roots and other parts of Aralia elata; saponins, especially Theasaponin E1 from the seeds of the tea plant (Camellia sinensis L).; extracts from bay leaf (Laurus nobilis), especially costunolide and its active component, alpha-methylene-gamma-butyrolactone (alpha-MGBL); proteinase inhibitor extracts from potato and soybean sources; a variety of oleanolic acid glycosides from many sources; also herbal combinations such as one consisting of yerba mate, damiana and guarana.

16. The method of claim 3 where the (−)-hydroxycitric acid is supplied as a therapeutically effective amount as the free acid, its lactone or as one or more of the salts or other derivatives of the free acid and is delivered in a controlled release form.

17. The method of claim 3 where the salts or derivatives are administered orally as a tablet or capsule wherein the contents of said capsule or tablet further comprise sodium bicarbonate, calcium carbonate, or potassium bicarbonate for producing carbon dioxide gas on contact with the stomach liquids wherein the amount of sodium bicarbonate, calcium carbonate or potassium bicarbonate is sufficient to cause the breakup of the capsule or tablet thus releasing the salts or derivatives, but insufficient to cause distension of the stomach.

18. The method of claim 3 where the salts or derivatives are administered orally as a tablet or capsule wherein the contents of said capsule or tablet further comprise sodium bicarbonate or potassium bicarbonate plus alginic acid; also capsules or tables containing sodium or potassium alginate.

19. The method of claim 3 where the salts or derivatives are administered orally as dry packaged powders designed to be mixed with water or juice and consumed between meals or prior to meals.

20. The method of claim 3 where the salts or derivatives are administered orally and are further encased in materials selected from the group consisting of gelatin, tapioca, gums, pectins, inulin, cellulose derivatives, alginic acid, dextran and dextrin for inclusion in thick drinks, soft-center bars and candies, pudding snacks, jelly-like confections, “gummy” deliveries and liquid meal replacements.

21. The method of claim 3 where the salts or derivatives are administered orally in conjunction with materials selected from the group consisting of citric acid, sodium or potassium citrate, other citric acid salts, sodium propionate, propionic acid, gallic acid, propyl gallate; extracts of marigold (Calendula officinalis); escins and other compounds from Aesculus hippocastanum seeds; extracts of the fruit of Kochia scoparia, and the roots and other parts of Aralia elata; saponins, especially Theasaponin E1 from the seeds of the tea plant (Camellia sinensis L).; extracts from bay leaf (Laurus nobilis), especially costunolide and its active component, alpha-methylene-gamma-butyrolactone (alpha-MGBL); proteinase inhibitor extracts from potato and soybean sources; a variety of oleanolic acid glycosides from many sources; also herbal combinations such as one consisting of yerba mate, damiana and guarana.

22-93. (canceled)
